(19) United States
(12) Patent Application Publication
(10) Pub. No.: US 2020/0399712 A1
(43) Pub. Date: Dec. 24, 2020

(54) COMPOSITIONS AND METHODS FOR THE DIAGNOSIS AND DETECTION OF TUMORS AND CANCER PROGNOSIS

(71) Applicant: The Regents of the University of California, Oakland, CA (US)

(72) Inventor: Joseph Califano, La Jolla, CA (US)

(21) Appl. No.: 17/011,732

(22) Filed: Sep. 3, 2020

Related U.S. Application Data
(63) Continuation of application No. PCT/US2019/020669, filed on Mar. 5, 2019.
(60) Provisional application No. 62/639,185, filed on Mar. 6, 2018.
(60) Provisional application No. 62/738,954, filed on Sep. 28, 2018.

(51) Int. Cl. C12Q 1/6886; C12Q 1/70

(52) U.S. Cl. CPC C12Q 1/6886; C12Q 2600/106; C12Q 2600/118; C12Q 1/708

(57) ABSTRACT

Provided herein are probe and primer pairs that are useful for the detection of HPV in samples isolated from patients.

### CROSS REFERENCE TO RELATED APPLICATIONS

This application claims priority under 35 U.S.C. § 120 to International PCT Application No. PCT/US2019/020669, filed Mar. 5, 2019, which in turn claims priority under 35 U.S.C. § 119(e) to U.S. Provisional Application No. 62/639,185, filed Mar. 6, 2018, and U.S. Provisional Application No. 62/738,954, filed Sep. 28, 2018, the content of each which is hereby incorporated by reference in its entirety.

### STATEMENT OF GOVERNMENT SUPPORT

This invention was made with Government support under CA211396 and DE023347 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Sep. 3, 2020, is named 114198-0391_SL.txt and is 16,628 bytes in size.

### BACKGROUND

Head and neck squamous cell carcinoma (HNSCC) is a highly lethal cancer that affects over 60,000 people in the US annually and has been traditionally associated with tobacco and ethanol exposure. Human Papilloma Virus (HPV) related HNSCC (HPVHNSCC) is known to occur in a separate patient population in comparison to tobacco related HNSCC, and also exhibits different etiology, site predilection, prognosis, as well as different genetic and epigenetic alterations in comparison to smoking related HNSCC. HPV is a ˜7.9 kb, non-enveloped, double-stranded, circular DNA virus that has a specific tropism for squamous epithelium, with HPV16 (p16+) as the most important subtype in the development of HNSCC. Regardless of the study population, high-risk HPV16 accounts for the overwhelming majority (95%) of HPV-positive tumors (21). The E6 and E7 oncoproteins coded within the viral genome are able to disrupt the function of Rb and p53, which are well known tumor suppressor genes, leading to development of a malignant phenotype. The traditional model of HPV carcinogenesis involves integration of a portion of the HPV genome into the host genome, with a focus on retention of E6 and E7 genes. However, recent data indicate that integration is identified only in about half of HPVOPC, and when integration does occur it can be in a non-uniform manner with variation in the size and region of HPV genome integration as well as host genome integration site (22).

Most of the HPV related tumors are primarily found in the oropharynx within the lingual and palatine tonsils, HPV positive oropharynx cancer (HPVOPC) patients are uniquely associated with p16 immunohistochemical (IHC) staining, more commonly male, white, non-smokers and non-drinkers and usually present at a more advanced stage (III/IV) at initial diagnosis. However, these patients have a significantly improved clinical prognosis and survival compared to non-HPV related HNSCC, although still with a 20% mortality at three years despite combined modality therapy (23). HPVOPC is not associated with the conventional HNSCC risk factors of tobacco and ethanol use, and instead is associated with lifetime exposure to sexual behaviors (i.e. number of lifetime vaginal and oral sex partners) and potentially with marijuana use (24-26).

While the overall incidence of many cancers, including HPV-unrelated oral cancers, has decreased in the past decades, the incidence rates of oropharyngeal cancer continues to increase (28-30), particularly among men under the age of 60. This increase has been observed both in the U.S. and abroad, and it is now clear that HPV infection is driving the increase in these rates (30). Incidence rates of oropharyngeal cancer in men have now exceeded the rates of cervical cancer in the United States (31).

HPVOPC has an improved survival as compared with HPV-negative OPC, documented in the landmark study RTOG 0129. This study examined outcomes in a trial of Stage III and IV squamous cell carcinoma from oral, pharyngeal, and laryngeal sites, randomizing to high dose cisplatin concurrently with accelerated fraction vs standard fractionation radiotherapy. A dramatic improvement in survival for HPVOPC was noted, with a 3-year progression free survival of 74% in HPV positive patients compared to 43% in HPV negative patients (23). In that analysis, the 3-year rate of local-regional recurrence was noted to be significantly lower in HPVOPC (14%) compared to HPV negative (35%), while distant metastasis was 9% and 15% respectively.

Thus a need exists in the art for accurate detection of HPV-DNA. This disclosure satisfies this need and provides related advantages as well.

### SUMMARY OF THE DISCLOSURE

This disclosure provides methods for treating an HPV-related cancer in a patient, the patient having been selected for the treatment by detection of one or more of HPV E2, E4 or E5 in a sample isolated from the patient, the method comprising, or consisting essentially of, or yet further consisting of administering an effective amount of one or more of: FGFR inhibitor AZD4547, radiotherapy, or cisplatin optionally with cetuximab, thereby treating the patient. Also provided herein are methods for detecting Human Papilloma Virus (HPV) in a sample comprising, or consisting essentially of, or yet further consisting of contacting the sample with the probe and primer pair described herein and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of the HPV, and detecting any HPV in the sample. In a further aspect, the presence of one or both of HPV E6 and E7 are tested, and one or both are determined to be absent.

This disclosure further provides methods for detecting Human Papilloma Virus (HPV)-related cancers in a sample comprising, or consisting essentially of, or yet further consisting of contacting the sample with the probe and primer pair described herein and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of the HPV, and detecting any HPV in the sample. In one aspect, the HPV-related cancer is the p16+ subtype.

Yet further provided are methods for monitoring disease progression in a cancer patient in remission from an HPV-related cancer, comprising, or consisting essentially of, or yet further consisting of contacting a sample isolated from the patient with the probe and primer pair of any one of this disclosure and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any HPV in the sample. Also disclosed are methods for predicting likelihood of clinical outcome or disease recurrence in a cancer patient, comprising, or consisting essentially of, or yet further consisting of contacting a sample isolated from the patient with the probe and primer pair of any one of this disclosure and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any HPV in the sample.

This disclosure also describes methods of determining whether a cancer patient will benefit from treatment with FGFR inhibitor AZD4547 radiotherapy, or cisplatin optionally with cetuximab, comprising, or consisting essentially of, or yet further consisting of contacting a sample isolated from the patient with the probe and primer pair provided herein, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any one of HPV E2, E4, or E5 and, optionally, not detecting any one of HPV E6 or E7 in the sample, wherein such detection indicates a cancer patient will benefit from treatment with FGFR inhibitor AZD4547, radiotherapy, or cisplatin optionally with cetuximab.

In one aspect of the above methods, the patient sample is negative for HPV E6 and E7. Thus, in another aspect, the methods further comprise, or consist essentially of, or yet further consist of detecting one or more of HPV E1, E2, E4, E5, E6 or E7 by contacting the isolated sample with a probe and primer pair of any one of:
a) a probe and primer pair detecting E1 selected from:
   i. a forward primer comprising, or consisting essentially of, or yet further consisting of the sequence: GCACAATTGGCAGACACTAAT (SEQ ID NO: 1), or an equivalent thereof,
   ii. a reverse primer comprising, or consisting essentially of, or yet further consisting of the sequence: GCACAATCCTTTACAATTTTTGCC (SEQ ID NO: 2), or an equivalent thereof, and
   iii. a probe comprising, or consisting essentially of, or yet further consisting of the sequence: AGTAATGCAAGTGCCTTTCTAAAAAGTAATTC (SEQ ID NO: 3), or an equivalent thereof,
b) a probe and primer pair detecting E1 selected from:
   i. a forward primer comprising, or consisting essentially of, or yet further consisting of the sequence: GCAAAACGCACAAAACGTGC (SEQ ID NO: 4), or an equivalent thereof,
   ii. a reverse primer comprising, or consisting essentially of, or yet further consisting of the sequence: GACATGTACCTGCCTGTTTGC (SEQ ID NO: 5), or an equivalent thereof, and
   iii. a probe comprising, or consisting essentially of, or yet further consisting of the sequence: CGGCTACCCAACTTTATAAAACA (SEQ ID NO: 6), or an equivalent thereof,
c) a probe and primer pair detecting one or more of HPV E1, E2, E4, E5, E6 or E7 comprising, or consisting essentially of, or yet further consisting of a sequence selected from those listed in Table 1 or an equivalent of each thereof,
d) a probe and primer pair that detects one of HPV E4 or an equivalent of each thereof, and
e) a probe and primer pair that detects one of HPV E5 or an equivalent of each thereof,
   under suitable conditions for detection of any HPV in the sample, and detecting any one of HPV E1, E2, E4, E5, E6 or E7 and, optionally, not detecting any one of HPV E6 or E7 in the sample.

In one embodiment, the sample is one or more of any area where HPV related cancers are known to develop, e.g., head and neck squamous cell carcinoma (HNSCC), HPV p16+ cancer, cancer of the skin, oropharynx, cancer of the pharyngeal, cancer of the cervix, cancer of the vulva, cancer of the penis, cancer of the anus, or cancer of the larynx. Additional samples include epithelia (skin) saliva, blood, plasma, or a tumor sample. In one particular aspect, the patient is a human patient. In another aspect, the HPV-related cancer is one or more of HPV-related head and neck squamous cell carcinoma (HNSCC), HPV p16+ cancer, cancer of the skin, oropharynx, cancer of the pharyngeal, cancer of the cervix, cancer of the vulva, cancer of the penis, cancer of the anus, or cancer of the larynx.

Further provided herein are the probes and primer pairs described above. In one embodiment, one or more of the forward primer, the reverse primer, or the probe described herein further comprise, or consist essentially of, or yet further consist of a detectable label. Also provided herein are kits for performing the methods as well as instructions for carrying out the methods of the present disclosure. The kit comprises, or alternatively consists essentially of, or yet further consists of one or more of probes and primer pairs of this disclosure and instructions for use.

### BRIEF DESCRIPTION OF THE DRAWINGS AND TABLES

FIGS. 1A-1D: HPV-16 DNA reads in each nucleotide position. Plots are shown for Applicants' cohort (FIG. 1A, FIG. 1C, FIG. 1D) and TCGA cohort (FIG. 1). (FIG. 1A), Average read counts in each HPV-16 nucleotide position in Applicants' 40 samples. It shows the highest read density (over 25000 reads) in the junction between E5 and L2 regions (nucleotide position: 4189-4227). (FIG. 1B), Average read counts in each HPV-16 nucleotide position in TCGA samples (N=94). It also shows the highest read density (over 40 reads) in the junction between E5 and L2 regions (nucleotide position: 4190-4261). (FIG. 1C), Minimum read counts in each HPV-16 nucleotide position in Applicants' all 40 samples. E1 specific region (nucleotide position: 1975-2195) contained the minimum read density across all samples. (FIG. 1D), After the exclusion of the lowest read sample data, minimum read counts replotted in each HPV-16 nucleotide position. Besides E1 specific region (MinR; nucleotide position: 1975-2195), E6 specific region (E6R; nucleotide position: 172-373) and the junction between E5 and L2 regions (MaxR; 4185-4334) also contained the minimum read density across these 39 samples.

FIGS. 2A-2F: Primer/probe design in these 3 specific regions and PCR amplifications. (FIG. 2A), In the specific region of E1, Applicants designed 6 candidate primer-probe sets (Min-1, 2, 3, 4, 5, and 6). Their amplicon lengths are 75 bp, 75 bp, 76 bp, 80 bp, 79 bp, and 78 bp, respectively. (FIG. 2B), PCR shows the reaction these 6 designed primers appropriately against DNA derived from Caski cell lines and does not show the reaction against DNA derived from lymphocyte of healthy individual. (FIG. 2C), In the region of junction between E5 and L2, Applicants designed 4 candidate primer-probe sets (Max-7,8,9, and 10). Their amplicon lengths are 70 bp, 70 bp, 75 bp, and 67 bp, respectively. (FIG. 2D), PCR shows the reaction these 4 designed primers appropriately against DNA derived from Caski cell lines and does not show the reaction against DNA derived from lymphocyte of healthy individual. (FIG. 2E), In the region of E6, Applicants designed 5 candidate primer-probe sets (E6-11,12,13,14, and 15). Their amplicon lengths are 80 bp, 81 bp, 85 bp, 85 bp, and 80 bp, respectively. (FIG. 2F), PCR shows the reaction these 5 designed primers appropriately against DNA derived from Caski cell lines and does not show the reaction against DNA derived from lymphocyte of healthy individual.

FIGS. 3A-3H: The sensitivity of primer-probe set and reaction under 30 and 10 copies of HPV-16 among DNA 10 ng derived from healthy individual. It shows the qPCR results of each primer-probe sets by the qPCR. All samples were tested 3 times. Of 15 planned primer-probe sets (see FIG. 2), there are 6 primer-probe sets which could detect 30 copies HPV-16 DNA among 10 ng normal human DNA. (FIG. 3A), E6 primer-probe sets. Under the 30 HPV-16 copies, there are 3 reactions, but only one reactions against 10 copies. (FIG. 3B), E7 primer-probe sets. Under the 30 HPV-16 copies, there are 3 reactions and 2 reactions against 10 copies. (FIG. 3C), “Min-4” primer-probe sets. Under the 30 HPV-16 copies, there are 3 reactions, and 2 reactions against 10 copies. (FIG. 3D), “Min-5” primer-probe sets. Under the 30 HPV-16 copies and 10 HPV-16 copies, there are also 3 reactions. (FIG. 3E), “Min-6” primer-probe sets. Under the 30 HPV-16 copies, there are 3 reactions, and 2 reactions against 10 copies. (FIG. 3F), “Max-10” primer-probe sets. Under the 30 HPV-16 copies, there are 3 reactions, but only one reactions against 10 copies. (FIG. 3G), “E6-11” primer-probe sets. Under the 30 HPV-16 copies, there are 3 reactions, but only one reactions against 10 copies. (FIG. 3H), “E6-13” primer-probe sets. Under the 30 HPV-16 copies and 10 HPV-16 copies, there are also 3 reactions.

FIGS. 4A-4B: Sensitivity of each primer-probe sets along with E6/E7 primer-probe sets against the 31 clinically known HPV-positive head and neck squamous cell carcinoma samples among DNA derived from healthy individual. (FIG. 4A), shows the sensitivity of samples against each planned primer-probe sets along with E6/E7 primer-probe sets. To calculate the threshold, Applicants prepared 32 clinically known HPV-positive head and neck squamous cell carcinoma anonymized samples which was made to dilution series to 150, 30, 15, 6, 1.2, 0.6, and 0.3 cells (correspond to 990 pg., 198 pg, 99 pg, 39.6 pg, 7.9 pg, 3.2 pg, and 1.6 pg of amount tumor weight, accordingly) among 10 ng normal human DNA. Each qPCR was studied 3 times, and the lowest dilution series which have 3 constant reactions was considered as threshold. Sensitivity was calculated as each 1) white, lower than 100 cells (correspond to 660 pg. of tumor weight), 2) gray, lower than 10 cells (correspond to 66 pg. of tumor weight), and 3) black, lower than 1 cell (correspond to 6.6 pg. of tumor weight). The named “Min-5” primer-probe sets were the highest sensitivity (28.1%) among lower than 1 cell in these 8 primer-probe sets. (FIG. 4B), combined with “Min-5” and “Max-10” primer-probe sets, the sensitivity is superior to E6 & E7 combined sensitivity according to all concentrations (<100 cells, <30 cells, <10 cells, <3 cells, <1 cell).

FIG. 5: Standard curve by RT-PCR of each primer-probe sets under the 20 ng DNA derived from HPV-HNSCC saliva samples. This figure shows the threshold and HPV-16 copy numbers of saliva samples by RT-PCR. “Max-10” primer-probe sets detect the remarkable more copy numbers than E6, E7, and “Min-5” primer-probe sets. “Max-10” primer-probe sets also showed the highest sensitivity among these 4 primer-probe sets. The slope of standard curve is −3.5 in “Min-5” primer-probe sets when delta Rn is plotted against cycle threshold.

FIG. 6: Sequence of the primer-probe sets of “5” (SEQ ID NOS 1-3, respectively, in order of appearance) and “10” (SEQ ID NOS 4-6, respectively, in order of appearance), respectively.

FIG. 7: Kaplan Meier curves for recurrence free survival based on 1-3 month post-treatment HPV16 presence in plasma, saliva, or both in previously untreated oropharynx cancer patients. See Ahn, S. M. et al. JAMA otolaryngology—head & neck surgery 140, 846-854, doi:10.1001/jamaoto.2014.1338 (2014). Saliva and plasma quantitative polymerase chain reaction-based detection and surveillance of human papillomavirus-related head and neck cancer.

FIGS. 8A-D: HPV E2, E4 and E5 genes expressions are associated with HPV integration. (FIGS. 8A-C), Heatmap of HPV genes expression (genes in rows and samples in columns). The expression levels of each gene in each sample was indicated gradual in grey scale. Samples were annotated in integration (darkest) and non-integration (white). According to the expression of E2, E4, E5 and E6, E7, there are two major clusters: most of samples in E2, E4, E5 high expression group were non-integrated, while samples in E6, E7 high expression group were integrated. (FIG. 8A), TCGA HPV+ HNSCC dataset. (FIG. 8B), TCGA CESC dataset. (FIG. 8C), Johns Hopkins HPV+ HNSCC dataset. (FIG. 8D), RT-qPCR validation of RNA-Seq results. 11 samples were chosen from Johns Hopkins HPV+ HNSCC dataset and HPV genes expression were validated with RT-qPCR using absolute quantitation method. The RT-qPCR result is completely concordant with RNA-Seq result.

FIG. 9: E2, E4, E5 attribute to cell proliferation in vitro and in vivo. Growth curve on the left and RT-qPCR validation on the right. Growth curve was measured by vita-blue method and normalized by the first day. Co-transfection of E4 siRNA and E6, E7 plasmid didn't show growth progress in HPV+ cervical cell line Caski, neither the co-transfection of E6 siRNA and E2, E4, E5 plasmid. Left histogram shows the effect of co-transfection. E2, E4, E5 were downregulated and E6, E7 were upregulated in E4 siRNA/E6, E7 plasmid group, while E6, E7 were downregulated and E2, E4, E5 were upregulated in E6 siRNA/E2, E4, E5 plasmid group. In vivo tumorigenesis activity of E2, E4, E5 in the CAL27 xenograft model. HPV-HNSCC cells CAL27/WT, CAL27/E245 and CAL27/E67 were transplanted to nude mice via subcutaneous injection. (data not shown). Tumor volumes were measured by caliper for indicated times.

FIGS. 10A-D: Effects of E2, E4, E5 on cell proliferation, cell cycle and apoptosis of HCT116 p53+/+ cells. HCT116 p53+/+ and HCT116 p53−/− E2/E4/E5 stable expression cell line were established. (FIG. 10A-B), On the left, proliferation was analyzed at 1, 2, 3, 4 and 5 days using vita-blue assay. Growth curves were normalized to the first day. On the right, stable expression of E2, E4, E5 were validated using RT-qPCR. (FIG. 10A), Relative viability in HCT116 p53+/+ cells. (FIG. 10B), Relative viability in HCT116 p53−/− cells. (FIG. 10C), Cell cycle was detected by FACS with PI staining in HCT116 p53+/+ cell line. On the left, representative cell cycle diagrams of control cells and irradiated HCT116 p53+/+ cells (6Gy) 24 hours after treatment. On the right, the percentages of different cell cycle phases using FlowJo. Error bars indicate the SD for 3 independent experiments. P values were calculated using Student t test. (FIG. 10D), Apoptosis was detected by FACS with PI and Annexin V-FITC staining in HCT116 p53+/+ cell line. On the left, representative apoptosis diagrams of control cells and irradiated HCT116 p53+/+ cells (6Gy) 48 hours after treatment. On the right, the percentages of apoptosis cells using FlowJo. Error bars indicate the SD for 3 independent experiments. P values were calculated using Student t test.

FIGS. 11A-B: HPV16 E2, E4, E5 and E6, E7 induced a marked skin hyperplasia and E2, E4, E5 accelerates tumorigenesis in a two-stage model of skin carcinogenesis. (FIG. 11A), Representative pictures of cK5-rtTA (control littermates) and cK5-rtTA/Tet-E2/E4/E5 animals 3 months after doxycycline induction. Fixed sections of wild-type (WT) and E2/E4/E5 mouse back skin were collected after doxycycline treatment (3 months) and stained with H&E. Quantification of the thickness of the epidermis (N=7). (FIG. 11B), Representative pictures of cK5-rtTA and cK5-rtTA/Tet-E6/E7 animals 3 months after doxycycline induction. Fixed sections of WT and E6/E7 mouse back skin were collected after doxycycline treatment and stained with H&E. Quantification of the thickness of the epidermis (N=7).

FIGS. 12A-B: Two subtypes share similar relative genes but distinguish from each other. (FIG. 12A) Onco-GPS map of E2, E4, E5 and E6, E7 subtypes and normal in significant gene sets, shown in grey scale. Each dark grey dot stands for one significant gene set, and each grey dot stands for one sample in E2, E4, E5 group (medium grey), E6, E7 group (dark grey) and normal (darker grey). A short distance from one sample to one gene set implied a close relationship between them. (FIG. 12B) Heatmap of significant gene sets depending on TCGA data. Five significant gene sets were chosen in each subtype. Two significant gene sets enriched in E2, E4, E5 subtype are both FGFR gene sets, and the high expression levels of these two gene sets are associated with non-integration.

FIG. 13: Unsupervised clustering of HPV+ HNSCC samples in TCGA dataset and JHH dataset using top 1000 genes depending on the SD among HPV+ HNSCC and normal samples. Annotations were made with HPV integration status and HPV genes subtypes. Heatmap of a subset of the TCGA samples.

FIG. 14: Similar to FIG. 13, unsupervised clustering of HPV+ HNSCC samples in TCGA dataset and JHH dataset using top 1000 genes depending on the SD among HPV+ HNSCC and normal samples. Annotations were made with HPV integration status and HPV genes subtypes. Heatmap of a subset of the JHH samples.

FIGS. 15A-B: DNA methylation and gene expression analysis with HPV+ HNSCC TCGA dataset. (FIG. 15A), Heatmap of DNA methylation levels for significantly differentially methylated CpG loci (rows) for all HPV-positive samples (columns). The columns of the heatmap are supervised by HPV genes expression subtypes: samples with E2, E4, E5 high expression are marked at the top of the heatmap in light grey, and samples with E6, E7 high expression are marked in dark grey. (FIG. 15B), Heat map of mRNA expression for significantly differentially expressed genes (rows) for all HPV-positive samples (columns). The columns of the heatmap are supervised by HPV genes expression subtypes.

FIGS. 16A-B: Representative alternated events in E2, E4, E5 and E6, E7 subtypes. Alteration events for top alteration genes are displayed by samples in (FIG. 16A) TCGA HPV+ HNSCC significant mutations and CNA in E2, E4, E5 and E6, E7 groups. (FIG. 16B) TCGA CESC significant mutations and CNA in E2, E4, E5 and E6, E7 groups. Two sided Fisher's exact test P values access the association of each genomic alteration in two subtypes.

FIGS. 17A-B: Association between HPV genes subtypes with disease-free survival in a separate HPV positive OPSCC cohort. (FIG. 17A), A disease-free survival analysis was performed using Prism 7. Recurrence time was defined to be the time in months from tumor biopsy to recurrence, death or loss to follow-up in three years. The log rank test was applied to test for the association of HPV subtypes and disease-free survival. There was no statistical association of HPV genes expression with clinical outcome (P=0.44). (FIG. 17B), HPV genes expression levels in two prognosis groups. 12 samples without recurrence in three years were defined as good prognosis cohort, and 11 samples with recurrence were defined as poor prognosis cohort. RT-qPCR using absolute quantitation method was performed to measure the expression levels of each HPV16 genes. Relatively low expression of E2, E4, E5 as well as E6, E7 was correlated with good prognosis.

FIGS. 18A-B: E2, E4, E5 and E6, E7 have no significant influence on cell growth in HOK and Detroit cells. Growth curve on the left and RT-qPCR validation on the right. Growth curve was measured by vita-blue method and normalized by the first day. Left histogram shows the effect of transfection validated with RT-qPCR. E2, E4, E5 and E6, E7 were upregulated in corresponding group. (FIG. 18A), Transfection of E2, E4, E5 plasmid or E6, E7 plasmid didn't show growth progress in HOK cells. (FIG. 18B), Transfection of E2, E4, E5 plasmid or E6, E7 plasmid didn't show growth progress in Detroit562 cells.

FIGS. 19A-B: No significant effects of E2, E4, E5 on cell cycle and apoptosis of HCT116 p53−/− cells were shown. HCT116 p53−/− E2/E4/E5 stable expression cell line were established. (FIG. 19A), Cell cycle was detected by FACS with PI staining. On the left, representative cell cycle diagrams of control cells and irradiated cells 24 hours after 6Gy irradiation. On the right, the percentages of different cell cycle phases using FlowJo. Error bars indicate the SD for 3 independent experiments. P values were calculated using Student t test. (FIG. 19B), Apoptosis was detected by FACS with PI and Annexin V-FITC staining. On the left, representative apoptosis diagrams of control cells and irradiated cells 48 hours after 6Gy irradiation. On the right, the percentages of apoptosis cells using FlowJo. Error bars indicate the SD for 3 independent experiments. P values were calculated using Student t test.

FIG. 20: A summary of assay development.

FIGS. 21A-D: HPV-16 DNA reads in each nucleotide position. Plots are shown for Applicants' cohort (FIG. 21A, FIG. 21C, FIG. 21D) and TCGA cohort (FIG. 21B). (FIG. 21A), Average read counts at each HPV-16 nucleotide position in 40 samples. The highest read density (over 25 000 reads) is observed at the junction between E5 and L2 regions (nucleotide position: 4189-4227). (FIG. 21B), Average read counts at each HPV-16 nucleotide position in TCGA samples (N=94). The highest read density (>40 reads) is observed at the junction between E5 and L2 regions (nucleotide position: 4190-4261). (FIG. 21C), Minimum read counts at each HPV-16 nucleotide position in all 40 samples. E1 specific region (nucleotide position: 1975-2195) contained the minimum read density across all samples. (FIG. 21D), After exclusion of the lowest read sample data, minimum read counts replotted in each HPV-16 nucleotide position. Besides E1 specific region (BR E1; nucleotide position: 1975-2195), E6 specific region (BR E6; nucleotide position: 172-373) and the junction between E5 and L2 regions (HR E5L2; 4185-4334) contained the minimum read density across these 39 samples.

FIGS. 22A-F: Primer/probe design in the three specific regions and PCR amplification. (FIG. 22A), In the specific region of E1, six candidate primer/probe sets (BR E1-1, 2, 3, 4, 5, and 6) of amplicon lengths 75 bp, 75 bp, 76 bp, 80 bp, 79 bp, and 78 bp, respectively were designed. (FIG. 22B), PCR shows that the six designed primers appropriately reacted against DNA derived from CaSki cell lines and did not react against DNA derived from lymphocyte of a healthy individual. (FIG. 22C), In the region of junction between E5 and L2, four candidate primer/probe sets (HR E5L2-1,2,3, and 4) of amplicon lengths 70 bp, 70 bp, 75 bp, and 67 bp, respectively were designed. (FIG. 22D), PCR shows that the four designed primers appropriately reacted against DNA derived from CaSki cell lines and did not react against DNA derived from lymphocyte of a healthy individual. (FIG. 22E), In the region of E6, five candidate primer/probe sets (BR E6-1,2,3,4, and 5) of amplicon lengths 80 bp, 81 bp, 85 bp, 85 bp, and 80 bp, respectively were designed. (FIG. 22F), PCR shows that the five designed primers appropriately reacted against DNA derived from CaSki cell lines and did not react against DNA derived from lymphocyte of a healthy individual. Each arrow indicates the primer location and the bold line shows the probe location.

FIGS. 23A-B: Sensitivity of each primer/probe sets along with that of E6/E7 primer/probe sets against the 31 clinically known HPV-positive head and neck squamous cell carcinoma samples among DNA derived from healthy individual. (FIG. 23A), Sensitivity of samples against each planned primer/probe sets along with E6/E7 primer/probe sets. To calculate the threshold, 32 clinically known HPV-positive head and neck squamous cell carcinoma anonymized samples were prepared that were serially diluted to 150, 30, 15, 6, 1.2, 0.6, and 0.3 cells (corresponding to 990 pg., 198 pg, 99 pg, 39.6 pg, 7.9 pg, 3.2 pg, and 1.6 pg of tumor weight, respectively) among 10-ng normal human DNA. Each qPCR was studied thrice, and the lowest dilution series showing three consistent reactions was considered as the threshold. Sensitivity was calculated as follows: 1) white, 100 cells (corresponds to 660 pg of tumor weight), 2) gray, 10 cells (corresponds to 66 pg of tumor weight), and 3) black, 1 cell (corresponds to 6.6 pg of tumor weight). The “BR E1-5” primer/probe sets showed the highest sensitivity (28.1%) for one cell among these eight primer/probe sets. (FIG. 23B), The sensitivity of the combination of “BR E1-5” and “HR E5L2-10” primer/probe sets was superior to that of E6 & E7 combination at all concentrations (100 cells, 30 cells, 10 cells, 3 cells, 1 cell). Experiments were performed in triplicates.

FIGS. 24A-B: Measurement range of HPV 16 CLIA test reagents in saliva. HPV-16 whole genome plasmid spiked into saliva. Arrows indicate upper and lower limits of quantification, accounting for precision and linearity. Data are plotted in copies/reaction (conversion to copies/mL, add 0.7 per replicate). Y axis=measured copies, X axis=expected copies a) HR E5L 2-4 assay, b) BR E1-5 assay. Similar results with E7 assay and SiHA DNA (not shown).

TABLE 1: This table provides the sequences of primers and probe set of HPV16 genes.

TABLE 2: This table provides performance characteristics of CLIA test for HPV16.

TABLE 3: This table provides predetermined metrics and actual results.

TABLE 4: This table provides pilot data regarding performance of CLIA HPV test in actual patient and control samples.

TABLE 5: This table summarizes the threshold of tumor cell numbers for HPV-16 detection. The column shown is the lowest tumor cell counts for the primer/probe for each sample set.

TABLE 6: This Table summarizes sensitivity from each tumor DNA dilution according to the prime/probe set.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

The practice of the present disclosure employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R.I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### MODES FOR CARRYING OUT THE DISCLOSURE

As explained in more detail herein, in one aspect, Applicants examined the risk of death after progression of HPVOPC with an analysis of patients with Stage III-IV OPC enrolled in RTOG 0129 and RTOG 0522 and a trial of accelerated fractionation radiotherapy and high dose cisplatin administered with or without weekly cetuximab (32). As provided herein, Applicants found that using Kaplan-Meier analysis, p16-positive (p16+) patients had significantly improved overall survival (OS) after disease progression when compared with p16-negative patients (hazard ratio [HR], 0.49; 95% CI, 0.34 to 0.70; P<0.001). Estimated median OS after progression was 2.6 years (95% CI, 1.5 to 5.1) versus 0.8 years (95% CI, 0.6 to 0.9) for p16-positive versus p16-negative patients, respectively. Estimated 2-year OS after progression was 54.6% for patients with p16-positive tumors (95% CI, 44.9% to 64.4%) and 27.6% for patients with p16-negative tumors (95% CI, 17.3% to 37.9%). When compared with patients with p16-negative tumors, patients with p16-positive tumors had an estimated 52% reduction in risk of death after TR adjustment for other factors (HR, 0.48; 95% CI, 0.31 to 0.74).

Patients with recurrent HPVOPC have an improved survival when compared to HPV negative OPC patients, and a majority of these patients have an opportunity for durable disease control. These data imply that effective early detection of recurrent disease in HPVOPC patients offers an opportunity to effectively salvage patients with recurrent/metastatic disease.

HPV16 subtype were selected as these are associated with approximately 95% of HPVHNSCC(9). Applicants employed two distinct datasets with whole genome sequencing of HPVHNSCC, followed by rational primer probe design based on read density across the HPV16 genome as well as presence in a high proportion of tumors, followed by analytic optimization and validation of performance in detection of HPV16 in salivary rinses from HPVHNSCC patients.

Applicants further identified an alternative mechanism of HPV 16 carcinogenesis driven by non-integrated HPV16 episomal HPV E2, E4 and E5 expression. Approximately half of HPV16 positive cervical and pharyngeal cancers comprise a distinct subtype with minimal expression of HPV16 E6 and E7, but dramatic increase in expression of E2, E4, and E5, as well as exclusive association of E2, E4, and E5 expression with lack of HPV16 integration into the host genome. Using cell line systems, Applicants demonstrated that concurrent E2/E4/E5 expression contributes to proliferation and impairs the G1-S DNA damage checkpoint in a p53 dependent manner. To evaluate the effect of E2, E4 and E5 expression in vivo, Applicants generated cK5-rtTA/Tet-E2/E4/E5 transgenic mice and observed an induction of hair loss and an increase in skin thickness that exceeded that in cK5-rtTA/Tet-E6/E7 transgenic mice. In a chemical carcinogenesis model, E2/E4/E5 mice were as or more susceptible to induction of cancer compared to E6/E7 mice. In addition, whole genomic network analysis indicated that the HPV16 E2/E4/E5 pharyngeal cancer subtype is characterized by fibroblast growth factor (FGR) pathway activation, and that this pathway may be targeted in HPV 16 tumors. This data demonstrates a common alternate mechanism of HPV viral oncogenesis that does not employ E6 and E7 expression or viral integration, and suggests that HPV related head and neck cancers may be driven by an alternate mechanism of episomal E2, E4, and E5 expression with implications for targeted therapy based on driver pathways associated with this subtype. These finding show that early detection and analysis of E2/E4/E5/E6 and E7 expression is useful for diagnosis and treatment of these cancers. This disclosure provides compositions and methods to aid in these objectives.

### Definitions

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “determining” or “identify” or “identifying” is to associate or affiliate a patient closely to a group or population of patients who likely experience the same or a similar clinical response to treatment.

“Detecting” as used herein refers to determining the presence of a nucleic acid of interest in a sample. Detection does not require the method to provide 100% sensitivity. Various means of detection are known in the art.

As used herein, the term “sample,” “test sample,” “test genomic sample” or “biological sample” refers to any liquid or solid material derived from an individual believed to have or having cancer. In some embodiments, a test sample is obtained from a biological source, such as cells in culture or a tissue or fluid sample from an animal, most preferably, a human. Exemplary samples include any sample containing the nucleic acid (e.g., DNA or RNA) of interest and include, but are not limited to, a tumor, a circulating tumor cell, cell free DNA (cfDNA), biopsy, aspirates, plasma, serum, whole blood, blood cells, lymphatic fluid, cerebrospinal fluid, synovial fluid, urine, saliva, and skin or other organs (e.g. biopsy material including tumor or bone marrow biopsy). The term “patient sample” as used herein may also refer to a tissue sample obtained from a human seeking diagnosis or treatment of cancer or a related condition or disease. It is also understood that these terms can encompass a population of purified cancer or pre-cancerous cells or a mixture of normal and cancer/precancerous cells. Each of these terms may be used interchangeably.

“Platinum drugs” refer to any anticancer compound that includes platinum. In an embodiment, the anticancer drug can be selected from cisplatin (cDDP or cis-iamminedichloroplatinum(II)), carboplatin, oxaliplatin, and combinations thereof.

“Oxaliplatin” (Eloxatin®) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumour activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

The phrase “first line” or “second line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

The term “adjuvant” chemotherapy refers to administration of a therapy or chemotherapeutic regimen to a patient after removal of a tumor by surgery. Adjuvant chemotherapy is typically given to minimize or prevent a possible cancer reoccurrence. Alternatively, “neoadjuvant” chemotherapy refers to administration of therapy or chemotherapeutic regimen before surgery, typically in an attempt to shrink the tumor prior to a surgical procedure to minimize the extent of tissue removed during the procedure.

As used herein, “having an increased risk” means a subject is identified as having a higher than normal chance of developing a cancer, compared to the average cancer patient. In addition, a subject who has had, or who currently has, cancer is a subject who has an increased risk for developing cancer, as such a subject may continue to develop cancer or metastatic disease. Subjects who currently have, or who have had, a tumor also have an increased risk for tumor metastases.

As used herein, “determining a prognosis” refers to the process in which the course or outcome of a condition in a patient is predicted. The term “prognosis” does not refer to the ability to predict the course or outcome of a condition with 100% accuracy. Instead, the term refers to identifying an increased or decreased probability that a certain course or outcome will occur in a patient exhibiting a given condition/marker, when compared to those individuals not exhibiting the condition. The nature of the prognosis is dependent upon the specific disease and the condition/marker being assessed. For example, a prognosis may be expressed as the amount of time a patient can be expected to survive, the likelihood that the disease goes into remission or experience recurrence, or to the amount of time the disease can be expected to remain in remission before recurrence.

A “blood cell” refers to any of the cells contained in blood. A blood cell is also referred to as an erythrocyte or leukocyte, or a blood corpuscle. Non-limiting examples of blood cells include white blood cells, red blood cells, and platelets.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively, the amplified sequence(s) may be cloned prior to sequence analysis. A method for the direct cloning and sequence analysis of enzymatically amplified genomic segments is known in the art.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this disclosure include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

When the probe and primer pairs are used as a basis or to aid in determination of, or for selecting a patient for a treatment described herein, the detection of HPV is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefit.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

“An effective amount” intends to indicated the amount of a compound or agent administered or delivered to the patient which is most likely to result in the desired response to treatment. The amount is empirically determined by the patient's clinical parameters including, but not limited to the stage of disease, age, gender, histology, and likelihood for tumor recurrence.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival (DFS), time to tumor recurrence (TTR), time to tumor progression (TTP), relative risk (RR), toxicity or side effects.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease.

“Disease free survival” (DFS) indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “stage I cancer,” “stage II cancer,” “stage III cancer,” and “stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. Seepages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

A “lymph node” refers to a rounded mass of lymphatic tissue that is surrounded by a capsule of connective tissue, which filter lymphatic fluid and stores white blood cells. Cancers described herein can spread to the lymphatic system and this spreading is used, in part, to determine the cancer stage. For example, if a cancer is “lymph node negative,” the cancer has not spread to the surrounding or nearby lymph nodes and thus the lymphatic system. Conversely, if the cancer has spread to the surrounding or nearby lymph nodes, the cancer is “lymph node positive.”

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

The term “hazard ratio” is a survival analysis in the effect of an explanatory variable on the hazard or risk of an event. In another aspect, “hazard ratio” is an estimate of relative risk, which is the risk of an event or development of a disease relative to treatment and in some aspects the expression levels of the gene of interest. Statistical methods for determining hazard ratio are well known in the art.

### Probe and Primer Pairs

Applicants provide herein a probe and primer pair, comprising, or consisting essentially of, or yet further consisting of:
a. a forward primer having the sequence: GCACAATTGGCAGACACTAAT (SEQ ID NO: 1), or an equivalent thereof,
b. a reverse primer having the sequence: GCACAATCCTTTACAATTTTTGCC (SEQ ID NO: 2), or an equivalent thereof, and
c. a probe having the sequence: AGTAATGCAAGTGCCTTTCTAAAAAGTAATTC (SEQ ID NO: 3), or an equivalent thereof. This probe and primer pair can be used to detect E1.

Also provided herein is a probe and primer pair, comprising, or consisting essentially of, or yet further consisting of:
a. forward primer having the sequence: GCAAAACGCACAAAACGTGC (SEQ ID NO: 4), or an equivalent thereof,
b. a reverse primer having the sequence: GACATGTACCTGCCTGTTTGC (SEQ ID NO: 5), or an equivalent thereof, and
c. a probe having the sequence: CGGCTACCCAACTTTATAAAACA (SEQ ID NO: 6), or an equivalent thereof, that in one aspect can be used to detect E4.

This disclosure further provides:
a) a probe and primer pair detecting E1 selected from:
   i. a forward primer comprising, or consisting essentially of, or yet further consisting of the sequence: GCACAATTGGCAGACACTAAT (SEQ ID NO: 1), or an equivalent thereof,
   ii. a reverse primer comprising, or consisting essentially of, or yet further consisting of the sequence: GCACAATCCTTTACAATTTTTGCC (SEQ ID NO: 2), or an equivalent thereof, and
   iii. a probe comprising, or consisting essentially of, or yet further consisting of the sequence: AGTAATGCAAGTGCCTTTCTAAAAAGTAATTC (SEQ ID NO: 3), or an equivalent thereof,
b) a probe and primer pair detecting E1 selected from:
   i. a forward primer comprising, or consisting essentially of, or yet further consisting of the sequence: GCAAAACGCACAAAACGTGC (SEQ ID NO: 4), or an equivalent thereof,
   ii. a reverse primer comprising, or consisting essentially of, or yet further consisting of the sequence: GACATGTACCTGCCTGTTTGC (SEQ ID NO: 5), or an equivalent thereof, and
   iii. a probe comprising, or consisting essentially of, or yet further consisting of the sequence: CGGCTACCCAACTTTATAAAACA (SEQ ID NO: 6), or an equivalent thereof,
c) a probe and primer pair detecting one or more of HPV E1, E2, E4, E5, E6 or E7 comprising, or consisting essentially of, or yet further consisting of a sequence selected from those listed in Table 1 or an equivalent of each thereof,
d) a probe and primer pair that detects one of HPV E4 or an equivalent of each thereof, and
   a probe and primer pair that detects one of HPV E5 or an equivalent of each thereof.

Further contemplated herein are primers and that detect HPV E2, E4, and/or E5, optionally for use in a PCR-based detection method. In certain aspects, these are used in conjunction with the primers and probes described herein above.

The individual probes and/or primers have as the minimum the sequences provided above or an equivalent thereof, as described herein. Minor changes to the primary sequences can be made, for example removal of 5′ and/or 3′ polynucleotides, as long as the sequences retain specificity to the target sequence, e.g., hybridization under conditions of high stringency. Additional 5′ and/or 3′ polynucleotides can be added to the probes and/or primers, e.g., the addition of polynucleotides that assist in hybridization to the target sequence under conditions of high stringency. For example, between 1 and 50, or between 1 and 40, or between 1 and 30, or between 1 and 20, or between 1 and 10, or between 1 and 5 (and ranges in between) can be added to either or both the 5′ and 3′ termini.

In one aspect, the pairs further comprise, or consist essentially of, or yet further consist of a detectable label on one or more of the forward primer, the reverse primer and the probe. Non-limiting examples include dyes, radioisotopes and fluorescent markers. One or both pairs can be packaged into kits with instructions for use.

In some embodiments, probes are labeled with two fluorescent dye molecules to form so-called “molecular beacons” (Tyagi, S. and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the disclosure also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837.

### Kits

As noted above, one or both the probes and/or primers can further comprise, or consist essentially of, or yet further consist of a detectable label on one or more of the forward primer, the reverse primer and/or the probe. One or both pairs can be packaged into kits with instructions for use. The kit can also comprise, or consist essentially of, or yet further consist of agents necessary for the preservation of those components comprised therein, e.g., a buffering agent, a preservative or a protein-stabilizing agent. The kit can further comprise, or consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components can be packaged in a manner customary for use by those of skill in the art. For example, these suggested kit components may be provided in solution or as a liquid dispersion or the like. Also provided herein are kits for performing the methods as well as instructions for carrying out the methods of the present disclosure. The kit comprises, or alternatively consists essentially of, or yet further consists of one or more of probes and primer pairs of this disclosure and instructions for use.

### Methods of Treatment, Detection Methods and Clinical Use

Provided herein are methods for treating an HPV-related cancer in a patient, the patient having been selected for the treatment by detection of one or more of HPV E2, E4 or E5, and optionally lack of detection of E6 and/or E7, in a sample isolated from the patient, the method comprising, or consisting essentially of, or yet further consisting of administering an effective amount of one or more of: FGFR inhibitor AZD4547, radiotherapy, or cisplatin optionally with cetuximab, thereby treating the patient.

Also provided herein are methods for detecting Human Papilloma Virus (HPV) in a sample comprising, or consisting essentially of, or yet further consisting of contacting the sample with the probe and primer pair described herein and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of the HPV, and detecting any HPV in the sample.

This disclosure further provides methods for detecting Human Papilloma Virus (HPV)-related cancers in a sample comprising, or consisting essentially of, or yet further consisting of contacting the sample with the probe and primer pair described herein and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of the HPV, and detecting any HPV in the sample.

Yet further provided are methods for monitoring disease progression in a cancer patient in remission from an HPV-related cancer, comprising, or consisting essentially of, or yet further consisting of contacting a sample isolated from the patient with the probe and primer pair of any one of this disclosure and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any HPV in the sample.

Also disclosed are methods for predicting likelihood of clinical outcome or disease recurrence in a cancer patient, comprising, or consisting essentially of, or yet further consisting of contacting a sample isolated from the patient with the probe and primer pair of any one of this disclosure and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any HPV in the sample.

This disclosure also describes methods of determining whether a cancer patient will benefit from treatment with FGFR inhibitor AZD4547 radiotherapy, or cisplatin optionally with cetuximab, comprising, or consisting essentially of, or yet further consisting of contacting a sample isolated from the patient with the probe and primer pair provided herein, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any one of HPV E2, E4, or E5 and, optionally, not detecting any one or both of HPV E6 or E7 in the sample, wherein such detection indicates a cancer patient will benefit from treatment with FGFR inhibitor AZD4547, radiotherapy, or cisplatin optionally with cetuximab.

In one aspect, the patient sample is negative for E6 and E7.

In another aspect the methods further comprise, or consist essentially of, or yet further consist of detecting one or more of HPV E1, E2, E4, E5, E6 or E7 by contacting the isolated sample with a probe and primer pair of any one of:
a) a probe and primer pair detecting E1 selected from:
   i. a forward primer comprising, or consisting essentially of, or yet further consisting of the sequence: GCACAATTGGCAGACACTAAT (SEQ ID NO: 1), or an equivalent thereof,
   ii. a reverse primer comprising, or consisting essentially of, or yet further consisting of the sequence: GCACAATCCTTTACAATTTTTGCC (SEQ ID NO: 2), or an equivalent thereof, and
   iii. a probe comprising, or consisting essentially of, or yet further consisting of the sequence: AGTAATGCAAGTGCCTTTCTAAAAAGTAATTC (SEQ ID NO: 3), or an equivalent thereof,
b) a probe and primer pair detecting E1 selected from:
   i. a forward primer comprising, or consisting essentially of, or yet further consisting of the sequence: GCAAAACGCACAAAACGTGC (SEQ ID NO: 4), or an equivalent thereof,
   ii. a reverse primer comprising, or consisting essentially of, or yet further consisting of the sequence: GACATGTACCTGCCTGTTTGC (SEQ ID NO: 5), or an equivalent thereof, and
   iii. a probe comprising, or consisting essentially of, or yet further consisting of the sequence: CGGCTACCCAACTTTATAAAACA (SEQ ID NO: 6), or an equivalent thereof,
c) a probe and primer pair detecting one or more of HPV E1, E2, E4, E5, E6 or E7 comprising, or consisting essentially of, or yet further consisting of a sequence selected from those listed in Table 1 or an equivalent of each thereof,
d) a probe and primer pair that detects one of HPV E4 or an equivalent of each thereof, and
e) a probe and primer pair that detects one of HPV E5 or an equivalent of each thereof,
   under suitable conditions for detection of any HPV in the sample, and detecting any one of HPV E1, E2, E4, E5, E6 or E7 and, optionally, not detecting any one of HPV E6 or E7 in the sample.

In one aspect, the assay is performed using the Min 5 and/or Min 10 probe/primer pairs disclosed herein.

In one embodiment, the sample is one or more of any tissue where HPV-related cancers are known to develop or have developed and include or include skin, saliva, blood, plasma, or a tumor sample. In one particular aspect, the patient is a human patient. In another aspect, the HPV-related cancer is one or more of HPV-related head and neck squamous cell carcinoma (HNSCC), skin cancer, HPV p16+, cancer of the oropharynx, cancer of the pharyngeal, cancer of the cervix, cancer of the vulva, cancer of the penis, cancer of the anus, or cancer of the larynx.

In one aspect, the treatment may be administered into a cavity formed by the resection of tumor tissue (i.e. intracavity delivery) or directly into a tumor prior to resection (i.e. intratumoral delivery). In some embodiments, the treatment may be administered intravenously, intrathecally, intraperitoneally, intramuscularly, subcutaneously, or by other suitable means of administration.

The treatments of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials. For the above methods, an effective amount is administered, when the administration of treatment serves to attenuate any symptom or prevent additional symptoms from arising. When administration is for the purposes of preventing or reducing the likelihood of cancer recurrence or metastasis, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

The methods also provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results, such as enhanced OS. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies e.g., surgical recession, chemotherapy, radiation. In one aspect, treatment excludes prophylaxis.

The probes and primer pairs are useful in a method for detecting Human Papilloma Virus (HPV) in a sample comprising, or consisting essentially of, or yet further consisting of contacting the sample with the probe and primer pair as described herein, or both, under suitable conditions for detection of the HPV, and detecting any HPV in the sample by detecting the probes bound to the target sequence. Non-limiting examples of samples are one or more of saliva, blood, plasma, or a tumor sample. Non-limiting examples of HPV-related cancers include without limitation is one or more of HPV-related head and neck squamous cell carcinoma (HNSCC), HPVp16+ cancer, cancer of the oropharynx, cancer of the pharyngeal, cancer of the cervix, cancer of the vulva, cancer of the penis, cancer of the anus, or cancer of the larynx. The cancer can be staged as Stage I, II, III or IV, and metastatic or non-metastatic.

Also provided herein is a method for detecting Human Papilloma Virus (HPV)-related cancers in a sample comprising, or consisting essentially of, or yet further consisting of contacting the sample with the probe or primer pair as described herein, or both, under suitable conditions for detection of the HPV, and detecting any HPV in the sample by detecting the probes bound to the target sequence. Non-limiting examples of samples are one or more of saliva, blood, plasma, or a tumor sample. Non-limiting examples of HPV-related cancers include without limitation is one or more of HPV-related head and neck squamous cell carcinoma (HNSCC), HPV p16+ cancer, skin cancer, cancer of the oropharynx, cancer of the pharyngeal, cancer of the cervix, cancer of the vulva, cancer of the penis, cancer of the anus, or cancer of the larynx. The cancer can be staged as Stage I, II, III or IV, and metastatic or non-metastatic

Further provided is a method for monitoring disease progression in a cancer patient in remission from an HPV-related cancer, comprising, or consisting essentially of, or yet further consisting of contacting a sample isolated from the patient with the probe and primer pair as described herein, or both, under suitable conditions for detection of any HPV in the sample, and detecting any HPV in the sample by detecting the probes bound to the target sequence. The method is repeated during and after treatment and compared to the results obtained previously. Non-limiting examples of HPV-related cancers include without limitation is one or more of HPV-related head and neck squamous cell carcinoma (HNSCC), HPVp16+ cancer, skin cancer, cancer of the oropharynx, cancer of the pharyngeal, cancer of the cervix, cancer of the vulva, cancer of the penis, cancer of the anus, or cancer of the larynx. Non-limiting examples of samples are one or more of saliva, blood, plasma, or a tumor sample. The cancer can be staged as Stage I, II, III or IV, and metastatic or non-metastatic.

Also provided is a method for predicting likelihood of clinical outcome or disease recurrence in a cancer patient, comprising, or consisting essentially of, or yet further consisting of contacting a sample isolated from the patient with the probe and primer pair as described herein, or both, under suitable conditions for detection of any HPV in the sample, and detecting any HPV in the sample by detecting the probes bound to the target sequence. In one aspect, the likelihood of a better clinical outcome or decreased likelihood of disease recurrence in a cancer patient is found when HPV is not detected in the sample. In another aspect, the likelihood of a worse clinical outcome or increased likelihood of disease recurrence in a cancer patient is found when HPV is detected in the sample. Non-limiting examples of samples are one or more of tissue where a HPV-related cancer is or may have developed, and include skin, saliva, blood, plasma, or a tumor sample. Non-limiting examples of HPV-related cancers include without limitation is one or more of HPV-related head and neck squamous cell carcinoma (HNSCC), HPV p16+, skin cancer of the oropharynx, cancer of the pharyngeal, cancer of the cervix, cancer of the vulva, cancer of the penis, cancer of the anus, or cancer of the larynx. The cancer can be staged as Stage I, II, III or IV, and metastatic or non-metastatic.

Still further aspects relate to a method of determining whether a cancer patient will benefit from the administration of FGFR inhibitor AZD4547, radiotherapy, or cisplatin optionally with cetuximab comprising, or consisting essentially of, or yet further consisting of contacting a sample isolated from the patient with the probe and primer pair as described herein, or both, under suitable conditions for detection of a variant of HPV in the sample susceptible to administration of FGFR inhibitor AZD4547, radiotherapy, or cisplatin optionally with cetuximab and detecting this HPV in the sample by detecting the probes bound to the target sequence. Non-limiting examples of samples include tissue where a HPV-related cancer is or may have developed, and include skin, saliva, blood, plasma, or a tumor sample. Non-limiting examples of HPV-related cancers include without limitation is one or more of HPV-related head and neck squamous cell carcinoma (HNSCC), HPV p16+ cancer, skin cancer, cancer of the oropharynx, cancer of the pharyngeal, cancer of the cervix, cancer of the vulva, cancer of the penis, cancer of the anus, or cancer of the larynx. The cancer can be staged as Stage I, II, III or IV, and metastatic or non-metastatic. In some embodiments, FGFR inhibitor AZD4547, radiotherapy, or cisplatin optionally with cetuximab is administered to the patient.

The methods are useful for the treatments of mammals and more particularly a human patient, e.g., a male or female patient.

The methods as described herein provide diagnostic, prognostic and therapeutic methods, which are based, at least in part, on determination of the presence of HPV in a tissue sample.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

Determining whether a subject is suitable or not suitable for cancer treatment of a given type, alternatively, can be expressed as identifying a subject suitable for the cancer treatment or identifying a subject not suitable for the cancer treatment of the given type.

It is to be understood that information obtained using the diagnostic assays described herein may be used alone or in combination with other information, such as, but not limited to, genotypes or expression levels of other genes, clinical chemical parameters, histopathological parameters, or age, gender and weight of the subject. For example, the tumor may be screened for p16 gene expression. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods are useful in the assistance of an animal, a mammal or yet further a human patient. For the purpose of illustration only, a mammal includes but is not limited to a simian, a murine, a bovine, an equine, a porcine or an ovine.

The methods described herein may be performed, for example, by utilizing pre-packaged diagnostic kits, such as those described below, comprising, or consisting essentially of, or yet further consisting of the probe and primer pairs described herein.

Sample nucleic acid for use in the above-described diagnostic and prognostic methods can be obtained from any suitable cell type or tissue of a subject. For example, a subject's bodily fluid (e.g. blood) can be obtained by known techniques (e.g., venipuncture and biopsy). Alternatively, nucleic acid tests can be performed on dry samples or preserved samples.

Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION. PROTOCOLS AND APPLICATIONS, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

The following examples are non-limiting and illustrative of procedures which can be used in various instances in carrying the disclosure into effect. Additionally, all reference disclosed herein are incorporated by reference in their entirety.

### Experimental Methods and Discussion

Human papillomavirus associated head and neck squamous cell carcinoma (HPV-HNSCC) is rapidly increasing in incidence and has unique epidemiologic, molecular, and biologic characteristics (1). Although HPV was only recently identified in HNSCC (2), HPV infection is a significant prognostic biomarker in HNSCC, especially in oropharyngeal carcinoma.

The presence of viral DNA as an indicator of disease presence has been used for multiple malignancies, including Epstein-Barr virus related nasopharyngeal carcinoma (4). HPV DNA detection in salivary rinses and/or plasma has also shown promise as a predictor of clinical behavior in HPV-related oropharyngeal carcinoma (HPV-OPC) (5). Improved detection of HPV DNA in HPV-OPC would potentially allow for improved assessment of disease burden and treatment response, and improved sensitivity of HPV-DNA based testing of salivary rinses and plasma may facilitate assessment of disease burden in HPV-OPC patients. In particular, this may allow early detection of HPV-OPC development or recurrence, with potential improvement in clinical outcomes.

Current PCR based detection methods for HPV are often based on historical, non-optimized primer-probe sets designed to identify genotype for high-risk HPV over 2 decades ago. These historical primer-probe sets also do not take advantage of recent whole genomic sequencing data, improved assay design techniques, and the benefits of analytic validation within a clinical testing environment. In the fragmented DNA from FFPE, the HPV detection by the historical primer-probe sets is sometimes difficult (6) due to amplicon size, and sensitivity can be challenging for low-copy number HPV-DNA detection in body fluids including saliva and plasma (7) (8). Optimizing primer-probe sets for HPVHNSCC would ideally maximize sensitivity of detection based on primer and probe design, as well as leverage the distribution and frequency of HPV genome in HPVHNSCC design to provide optimal detection of highly represented regions of tumor.

### Experiment No. 1

### Patient Characteristics

Primary tumor tissue samples were obtained from a cohort of 72 patients with HPV-related oropharyngeal squamous cell carcinoma, as previously described (10). Normal oropharynx tissue from uvulopharyngoplasty (UPPP) surgical specimens were obtained from 25 cancer unaffected controls. All tissue samples were collected from the Johns Hopkins Tissue Core under an approved IRB protocol (#NA_00-36235).

### Analysis of Sequencing Data

DNA was extracted from frozen tissue sections from 40 tissue samples and DNA sequencing was performed as previously described. Briefly, sequencing was performed using the HiSeq 2500 platform sequencer and the TruSeq Cluster Kit (Illumina), resulting in approximately 80 million paired reads per sample. An in-house pipeline was developed to extract all unmapped reads from BAM files and re-align them to HPV-16 genomes (accession number: AY686584.1). This pipeline includes the following steps: prepare HPV reference genome file, perform quality control on BAM files, extract unmapped read pairs, convert unmapped read pairs to FASTQ format, align unmapped read pairs to the HPV reference genomes (accession number: AY686584.1) by Rsamtools and then apply scanBam to each of them. Finally, only the aligned reads were filtered out and coverage function was applied.

### TCGA Data for Validation

Publically available TCGA WGS data generated using Illumina Infinium HumanMethylation450 (HM450K) BeadChip and clinical data were downloaded. All unmapped reads had realigned them to HPV-16 genomes (accession number: AY686584.1). As described above. WGS was performed on a separate Johns Hopkins cohort previously published, and publicly available data employed for further analysis.

### Primer-Probe Design

Primer sets were designed by Primer-BLAST in NCBI website (url: https://www.ncbi.nlm.nih.gov/tools/primer-blast). Applicants entered the HPV-16 accession number with focused regions, and constructed the PCR product size as small as possible, less than 85 bp and subsequently performed probe design. Probes were designed to be less than 35 bp, with an annealing temperature below 60° C. Specific primers and probes previously designed to amplify the E6 and E7 regions of HPV-16 were used as positive control. E6 forward primer, 5′-TCAGGACCCACAGGAGCG-3′ (SEQ ID NO: 7); E6reverse primer, 5′-CCTCACGTCGCAGTAACTGTTG-3′ (SEQ ID NO: 8); E6 Taqman probe, 5′-(FAM)-CCCAGAAAGTTACCACAGTTATGCACAGAGCT-(TAMRA)-3′ (SEQ ID NO: 9). E7 forward primer, 5′-CCGGACAGAGCCCATTACAA-3′ (SEQ ID NO: 10); E7 reverse primer, 5′-CGAATGTCTACGTGTGTGCTTTG-3′ (SEQ ID NO: 11); E7 Taqman probe, 5′-(FAM)-CGCACAACCGAAGCGTAGAGTCACACT-(TAMRA)-3′ (SEQ ID NO: 12).

### Polymerase Chain Reaction (PCR)

PCR was performed on DNA derived CaSki (American Type Culture Collection) cell line using designed primer sets using JumpStart REDTaq® ReadyMix (SIGMA-Aldrich, MO). This cell line is known to have 600 copies per genome of HPV-16 DNA. RNase free water was made and 0.2 M of each primer (forward and reverse) was added to the corresponding master mix. The amplification consisted of a denaturing phase at 95° C. for 3 min, 35 cycles of 95° C. for 30 s followed by 55° C. for 30 s and 72° C. for 1 min, then an elongation phase for 5 min at 72° C. All samples were stored at 4° C. Each PCR product was subsequently run on a 2.0% agarose gel via gel to evaluate PCR success.

### Quantitative Real-Time PCR

Quantitative real-time polymerase chain reaction (qRT-PCR) was used to examine the threshold in each experiment. qRT-PCR was performed using Quant Studio 6 Flex Real-Time PCR System (Thermo Fisher Scientific). All samples were run in triplicates. HPV-16 viral amplicon copy number estimations were developed using the CaSki cell line. After amplification of the amplicon by PCR, DNA concentration was calculated by weight NanoDrop®, then converted to DNA copy number by calculation based on the molecular weight of the double stranded amplicon. This known-copy number DNA was serially diluted into 100 copies, 30 copies, and 10 copies among 10 ng normal human DNA. qRT-PCR was performed and regarded as positive by consistent detection in all reactions in a triplicate with appropriate negative controls. In the other 32 HPV-HNSCC cohort, Applicants diluted the tumor sample to 990 pg, 198 pg, 99 pg, 39.6 pg, 7.9 pg, 3.2 pg, and 1.6 pg of amount tumor weight, (correspond to 150, 30, 15, 6, 1.2, 0.6, and 0.3 cells: assumption that 1 tumor cell have 6.6 pg DNA (=3*10\hat{ }9 (human DNA genome bp)*2 (diploid)*1.67*10\hat{ }(−24) g (weight/Dalton))) combined with 10 ng normal leukocyte human DNA and compare the threshold of each primer-probe sets. The lowest concentration that demonstrated consistent amplification was defined as the threshold for detection. In the 28 HPV-HNSC cohort, Applicants extracted DNA from saliva samples as previously described and seriously diluted to 20 ng and compare the copy-number of HPV-16 as previously described.

### Results

### HPV-16 Read Coverage and the Universal Regions Among HPV-HNSCC

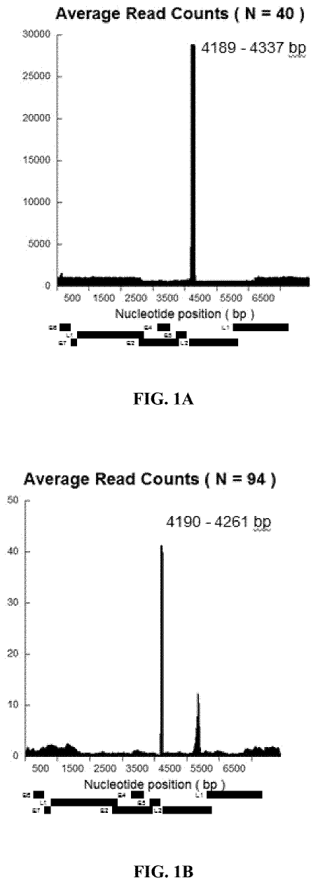

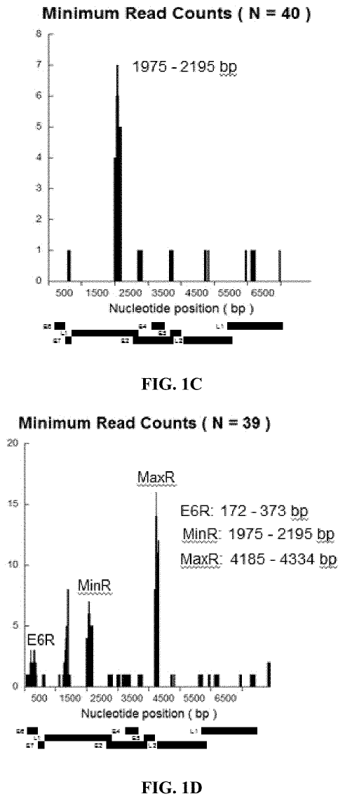

To determine the distribution of reads of HPV-16 genome in HPV-HNSCC, Applicants performed the realignment of each HPV-16 nucleotide on 40 HPV-HNSCC tumors. Utilizing WGS data Applicants conducted the column of each read coverage in each nucleotide position. Total read counts of HPV-16 genome were 452,590,608 reads (average per nucleotide: 1427 reads). Applicants first analyzed that average read counts in each nucleotide position of all 40 patients. There are remarkable spikes in the regions between 4189 bp to 4337 bp and this spike surpass 25,000 reads. This means that the regions between E5 and L2 noncoding area was the most remarkably frequent read coverage area (MaxR) in the HPV-16 genome (FIG. 1A).

To validate these findings, Applicants analyzed 94 HPV-HNSCC available from TCGA cohort. Applicants also conducted the column of each read coverage in each nucleotide position. Total read counts of HPV-16 genome were 166,026 reads (average per nucleotide: 3.70 reads). Applicants also analyzed that average read counts in each nucleotide position of these 94 patients. There are also remarkable spikes in the regions between 4190 bp to 4261 bp and this spike surpass 40 reads. These observations were consistent with MaxR derived from Applicants' cohort (FIG. 1).

Applicants then tried to define regions where HPV-16 genome was present in a minimal detectable level in all 40 tumor samples. Of 40 tumor samples, minimum read counts of each nucleotide position were plotted. The previous described spike regions between E5 and L2 is not a universal region of HPV-16 nucleotide. However, Applicants were able to define a region between 1975 bp to 2195 bp in the E1 regions for which every tumor analyzed contained at least 4 read counts (MinR) (FIG. 1C). Applicants could not confirm these observations by TCGA because of low read coverage data.

To define additional where HPV-16 genome was present in a minimal detectable level in a large proportion of tumors, Applicants omitted the lowest read coverage one sample (3.92 reads per nucleotide sample), then minimum read counts of each nucleotide position in the other 39 tumor samples were replotted. The MiR remained the universal regions, and the previously described MaxR along with the entire E6 (E6R), known as oncogene of HPV-16, was also present in 39/40 tumor samples (FIG. 1D).

These 3 regions were the promising candidate universal regions of HPV-HNSCC. Applicants focus these regions of E6R: 172-373 bp, MinR: 1975-2195 bp, and MaxR: 4185-4334 bp and try to design the primer-probe sets.

### Primer-Probe Design and Validation by PCR

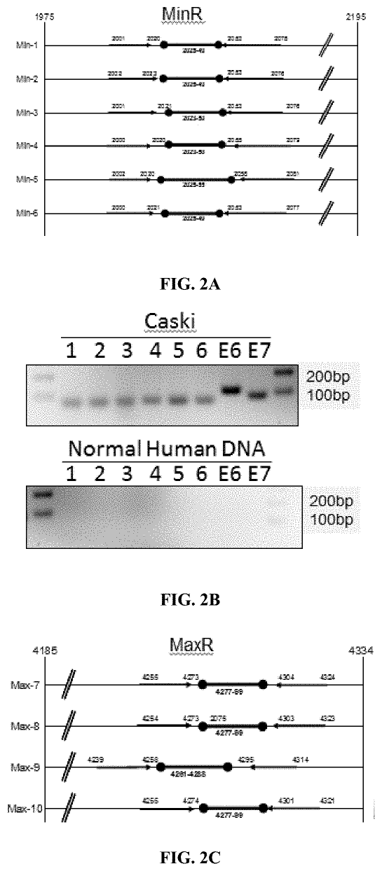

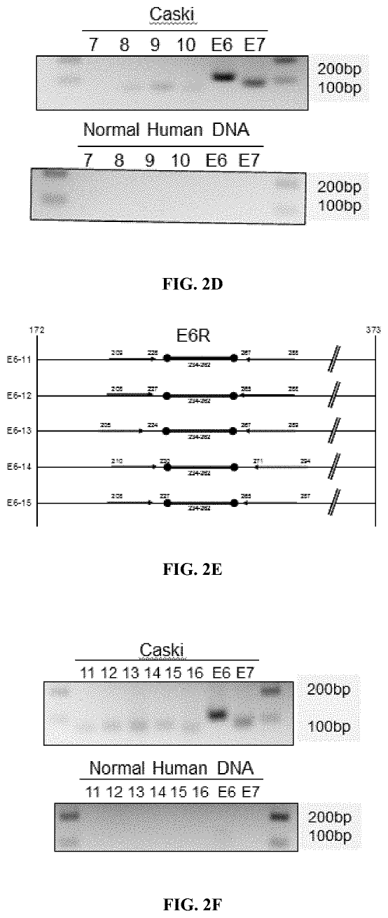

Next, Applicants sought to make the optimized primer-probe sets for these three regions. After the designing by Primer-BLAST, Applicants confirm the eligibility of the probe design, and the candidate primer-probe sets were constructed. In the “MinR” regions (between 1975 bp-2195 bp), there are 6 candidate primer-probe sets (named from “Min-1” to “Min-6”) which amplicon were all less than 80 bp (FIG. 2A). Applicants confirm the positive PCR reaction against DNA derived from Caski cell-line and the negative PCR reaction against DNA derived from normal human normal DNA along with existence E6, E7 primer sets (FIG. 2B). In the MaxR (between 4185 bp-4334 bp), there are 4 candidate primer-probe sets (named from “Max-7” to “Max-10”) which amplicon were also less than 80 bp (FIG. 2C). Applicants confirm the positive PCR reaction against DNA derived from Caski cell-line and the negative PCR reaction against DNA derived from normal human normal DNA along with existence E6, E7 primer sets (FIG. 2D). In the E6R (between 172 bp-373 bp), there are 5 candidate primer-probe sets (named from “E6-11” to “E6-15”) which amplicon were also less than 80 bp (FIG. 2E). Applicants confirm the positive PCR reaction against DNA derived from Caski cell-line and the negative PCR reaction against DNA derived from normal human normal DNA along with existence E6, E7 primer sets (FIG. 2F). Applicants cannot make the appropriate primer-probe sets other than the minimum read coverage area in the area of E7, E2 and L1 regions (FIG. 1C).

### Sensitivity of Primer-Probe Sets Against HPV-16 Copy Number

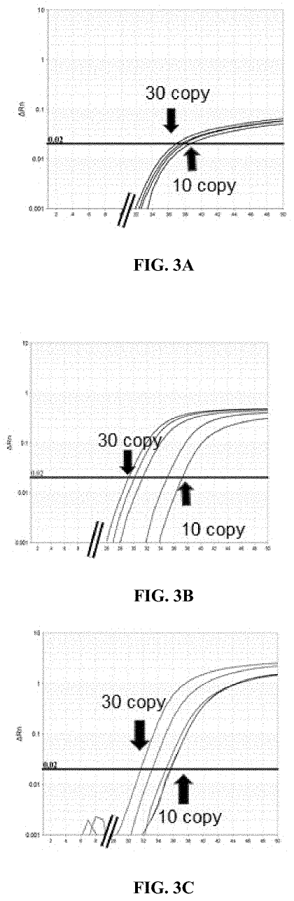

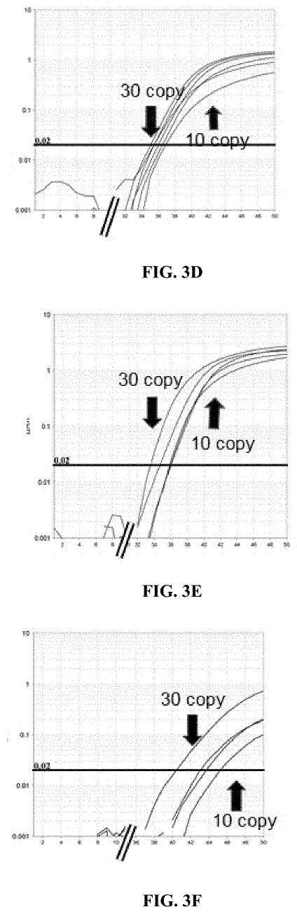

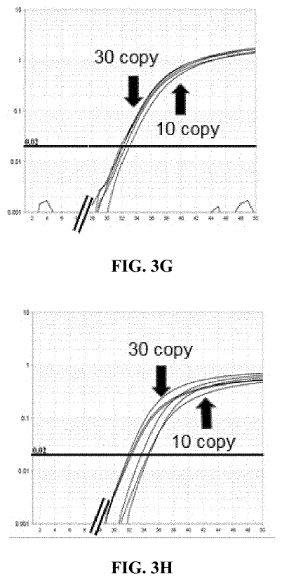

To seek the most promising primer-probe set in the viewpoint of sensitivity, Applicants evaluated the sensitivity analysis for these candidate primer-probe sets. First, Applicants confirmed the sensitivity of existing E6 and E7 primer-probe sets. Both of E6 and E7 primer-probe sets reacted consistently triplicate under 30 copies of HPV-16 genome among normal 10 ng DNA samples, but reacted inconsistently under 10 copies of HPV-16 genome. Therefore, the sensitivity of E6 (FIG. 3A) and E7 (FIG. 3B) was also 30 copies. Of 15 designed primer-probe sets, there are 6 primer-probe sets which react consistently under 30 copies of HPV-16 genome. The candidate primer-probe sets were the “Min-4” (FIG. 3C), “Min-5” (FIG. 3D), “Min-6” (FIG. 3E), “Max-10” (FIG. 3F), “E6-11” (FIG. 3G), “E6-13” (FIG. 3H), respectively. Notably, there remained consistently triplicate reaction under 10 copies in the “Min-5” primer-probe sets.

### Sensitivity of Primer-Probe Sets Against Tumor Samples

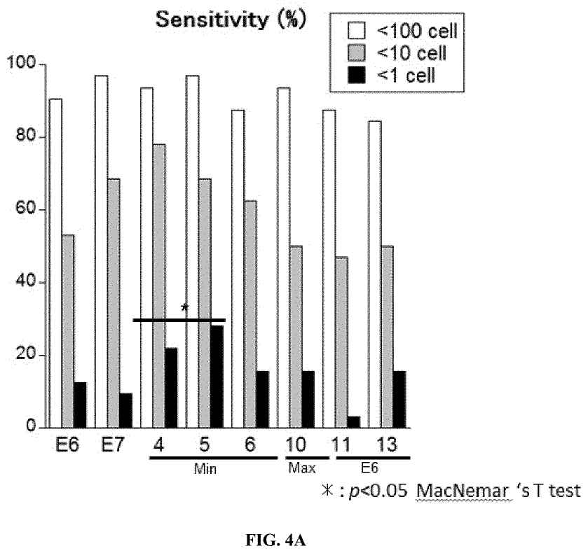

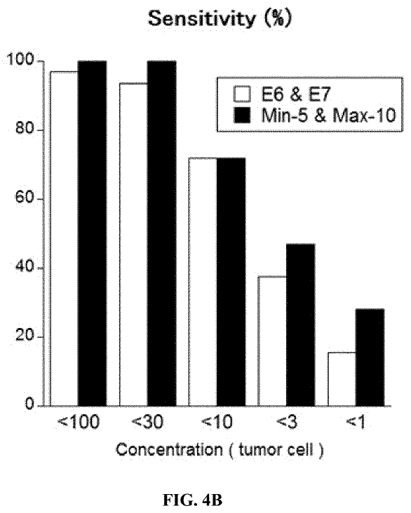

Applicants extended Applicants' findings to determine whether this new designed prime-probe sets also have high sensitivity to other tumor samples. In this regard, Applicants investigated the other 32 HPV-HNSCC tumor samples. From the assumption that 1 cell tumor DNA contain 6.6 pg DNA, Applicants made the dilution series by DNA derived from normal lymphocyte as described and decide the threshold for each primer-probe set. In this study, sensitivity under 100 cells (660 g DNA) was 90.6% (29/32) of E6 primer-probe sets, 96.9% (31/32) of E7 primer-probe sets, 93.8% (30/32) of “Min-4” primer-probe sets, 96.9% (31/32) of “Min-5” primer-probe sets, 87.5% (28/32) of “Min-6” primer-probe sets, 93.8% (30/32) of “Max-10” primer-probe sets, 87.5% (28/32) of “E6-11” primer-probe sets, and 84.4% (27/32) of “E6-13” primer-probe sets, respectively. In addition, sensitivity under 10 cells (66 pg DNA) was 53.1% (17/32) of E6 primer-probe sets, 68.8% (22/32) of E7 primer-probe sets, 78.1% (25/32) of “Min-4” primer-probe sets, 68.7% (22/32) of “Min-5” primer-probe sets, 62.5% (20/32) of “Min-6” primer-probe sets, 50.0% (16/32) of “Max-10” primer-probe sets, 46.9% (15/32) of “E6-11” primer-probe sets, and 50.0% (16/32) of “E6-13” primer-probe sets, respectively. Furthermore, sensitivity under single cells (6.6 pg DNA) was 12.5% (4/32) of E6 primer-probe sets, 9.4% (3/32) of E7 primer-probe sets, 21.9% (7/32) of “Min-4” primer-probe sets, 28.1% (9/32) of “Min-5” primer-probe sets, 15.6% (5/32) of “Min-6” primer-probe sets, 15.6% (5/32) of “Max-10” primer-probe sets, 3.1% (1/32) of “E6-11” primer-probe sets, and 15.6% (5/32) of “E6-13” primer-probe sets, respectively. By McNamar chi-square test, there is a significant difference between E7 and “Min-5” primer-probe sets under the single cell detection sensitivity (FIG. 4A). Furthermore, the sensitivity by combination with “Min-5” and “Max-10” primer-probe were higher than that of E6 and E7 primer-probe sets (FIG. 4B).

### Sensitivity of Primer-Probe Sets Against Saliva Samples

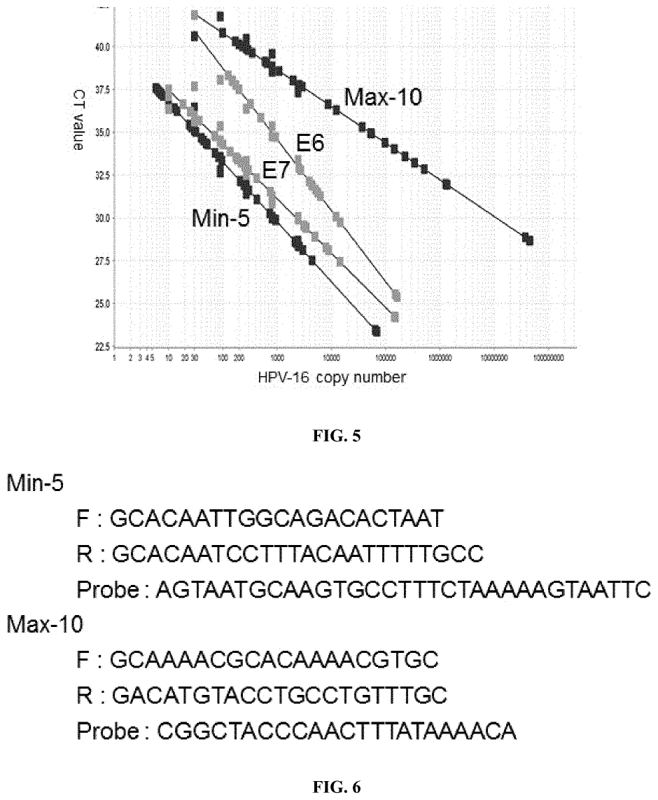

To seek the promising primer-probe set also react against the saliva samples, Applicants evaluated the sensitivity analysis for “Min-5” and “Max-10” primer-probe sets along with E6 and E7 primer-probe sets in the saliva DNA samples. The sensitivity of detection from saliva 20 ng DNA samples is 32.1% (9/28) in E6 primer-probe sets, 42.9% (12/28) in E7 primer-probe sets, 35.7% (10/28) in “Min-5” primer-probe sets, 46.4% (13/28) in “Max-10” primer-probe sets. The sensitivity of combination of “5” and “10” is 50% (14/28) and that of E6 and E7 is 42.9% (12/28). By the standard curve analysis, the copy numbers that detected by “Max-10” primer-probe sets were the highest in these 4 primer-probe sets. The slope of standard curve is −3.5 in “Min-5” primer-probe sets when delta Rn is plotted against cycle threshold (FIG. 5).

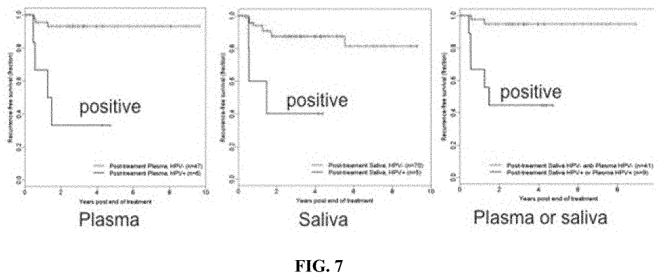

Finally, Applicants concluded that the combination with “Min-5” and “Max-10” primer-probe sets was the most promising optimized primer-probe set in this study because of its sensitivity for qPCR reaction against HPV-16 copy number along with tumor and saliva samples. Applicants showed the sequence (FIG. 6).

### Discussion

In this study, Applicants constructed the optimized new primer-probe set by rational approach from the deep sequence data with validation by TCGA. In addition, Applicants refined these primer-probe sets carefully by low copy number HPV and PV-HNSCC tumor samples detection among DNA derived from normal lymphocyte, because Applicants want to show the possibility of detection in the tiny DNA among saliva or plasma. Therefore, Applicants believe Applicants could determine the most optimized primer-probe sets from these well-designed preclinical experiments.

There are imitable differences between Applicants' designed primer-probe sets and historical primer-probe sets. First, historical primer set, known as “consensus” primer-sets, named “My09/MY11”, “GP5+/GP6+” et al. are not only specific to HPV-16 sequence but to detect many kinds of high-risk HPV (11), so the sensitivity for HPV-16 is not satisfactory. In addition, because their lengths of amplicon are over 150 bp (12), they are not suitable for screening from fragmented DNA, especially formalin-fixed specimen. Second, Applicants made the new primer-probe sets which have more sensitive than now widely used primer-probe set of E6 or E7. E6 and E7 are famous for their oncogenic role in HPV-HNSCC, but there is no consensus primer location for their nucleotide. Applicants first showed that the rational primer location which is not only mere oncogene, but the universal region among HPV-HNSCC from the result of high-throughput sequence data from HPV-HNSCC.

Applicants focused on the three universal regions which Applicants' 39 cases have the read. The functions of these regions are not obscure, especially among E1 region “MinR” and between E5 and L2 regions “MaxR”. Kalu N N et al. (13) reported the similar spikes of read coverage presented from HPV related tumor cell-line analysis, and Nulton T J et al. (14) also reported the detailed HPV genome status in HNSCC from TCGA database. In some cases, the remarkable high copy numbers of HPV-16 were also detected by “Max-10” primer-probe set in the saliva DNA samples, this indicates that the HPV-16 DNA spikes between T5 and L2 also present in at least saliva samples as FIGS. 1A and 1B presented. It is not sure that the spikes and universal regions in HPV-16 genome are unique for HNSCC or consensus for the other HPV-related carcinoma, including cervical carcinoma.

From the best of Applicants' knowledge, there are no reports according to the universal locations in the HPV-16 genome, named “MinR”. By Primer-BLAST designing technique, Applicants succeeded the primer-probe design “Min-5” which could detect under the 10 copy-HPV among the 10 ng DNA derived from normal lymphocyte, which also show the concordance that this “Min-5” primer-probe set also show the most sensitive under the validating tumor samples. The combination of “Min-5” and “Max-10” are primer-probe sets that can be used as a substitute for the E6 and E7 existing primer-probe sets.

In clinical practice, there are a new category of the HPV-related oropharyngeal carcinoma section in the AJCC 8^{th} classification (15). The definition of HPV positivity is ISH-HPV positivity and/or p16 overexpression, and there remains the problem about the tumor which shows p16 positive, but ISH-HPV negative (16). In clinical, there are some reports (17) that p16 positive tumor could show the favorable prognosis irrespective of HPV, but the reason is not obscure. Applicants' optimized designed primer-probe sets might answer this clinically undecided p16 positive-HPV negative tumor by improving the sensitivity of PCR detection.

Recently, digital PCR (dPCR) developed and showed a superior performance (18) to RT-PCR regarding reproductivity against especially low templete numbers. If Applicants explored the dPCR, the sensitivity itself might be higher than Applicants' RT-PCR results. However, it does not mean the worth of Applicants' designed primer-probe sets deteriorated, because Applicants rely on the reproductivity of triplicate determination in Applicants' RT-PCR results and the standard curve methods. dPCR also have several advances, especially it could not disturbed by PCR inhibitor (19) contained in clinical samples, so it would be a good approach for the detection by dPCR in the tiny DNA amount from the serum like circulating tumor cells, with Applicants' designed new primer-probe sets.

In conclusion, by the sequence-based rational approach, Applicants could extract new optimized primer-probe set for the detection HPV-16 for HNSCC.

### Experiment No. 2

HPV DNA based detection of HPVOPC. The presence of viral DNA as an indicator of disease presence has been used for multiple malignancies, including Epstein Barr virus related nasopharyngeal carcinoma (33). Despite barriers to HPV DNA based population screening for HPVHNSCC, detection of the HPV viral genome has been useful in a variety of other clinical situations. Molecular detection of HPV in metastatic cervical lymph nodes is a highly effective strategy for localizing the site of tumor origin to the oropharynx, and is now commonly used in clinical practice as an indicator for additional diagnostic and interventional strategies to localize occult primary OPC (34).

HPV DNA detection in salivary rinses has also shown promise as a predictor of clinical behavior in HPVOPC. Applicants performed a pilot study using fresh tumor samples and pre- and post-treatment salivary rinses from 59 patients with HNSCC. HPV-16 E6 and E7 DNA copy number in these samples were quantified by real time PCR. Twenty of 59 patients (33.9%) were HPV-16 positive in their tumors before treatment. Four of 20 HPV tumor positive patients ultimately developed recurrence, and two of these four patients were HPV-16 positive in surveillance salivary rinses (sensitivity=50%). Of the 39 (66.1%) HPV-16 negative patients on initial clinical presentation and the 16 HPV-16 positive patients who did not recur, none were HPV-16 positive in salivary rinses after treatment (specificity=100%) (35). These data demonstrate that HPV detection in salivary rinses in HPVOPC patients may be a promising means to detect and potentially predict recurrence.

In a similarly sized cohort, Cao et al. found that HPV DNA in plasma was found essentially exclusively in 65% of the HPVOPC patients, became undetectable with disease resolution, and similarly was able to define a cohort of patients with ultimate development of distant metastasis who demonstrated subsequent reappearance of HPV DNA in plasma samples (36).

### Detection of HPV DNA in Saliva and Plasma is a Biomarker for Disease Progression, Poor Outcome, and Precedes Clinical Detection of Recurrence

Based on data above, Applicants performed a follow up study in which 81 patients with HPVOPC were sampled for salivary rinses and plasma before and after treatment and analyzed for the presence of HPV16 DNA in E6 and E7 coding regions (20). The sensitivities of pretreatment saliva or plasma alone for defining HPVOPC were 52.8% and 67.3%, respectively. With a median follow up of 49 months (range, 0.9 to 181 months), the sensitivity, specificity, negative predictive value, and positive predictive value of combined saliva and plasma pretreatment.

HPV16 DNA status for detecting tumor HPV16 status were 76%, 100%, 42%, and 100%, respectively. Post treatment values showed 95% specificity for detection of recurrence with 55% sensitivity for plasma, with an increase in sensitivity to 69% for combined salivary rinse and plasma detection.

On multivariable analysis posttreatment saliva HPV DNA positive, plasma HPV DNA positive, and either plasma or saliva HPV DNA positive patients demonstrated an increased risk of disease recurrence, (HR, 10.7; 95% CI, 2.4-48.5, P=0.002). (HR, 41.5; 95% CI, 4.2-407.0, P=0.001). (HR, 24.4; 95% CI, 3.6-166.0, P=0.001) FIG. 7. In all, approximately half of all recurrences were associated with post-treatment plasma or saliva DNA detection prior to diagnosis of recurrence. The lead time for saliva rinse based detection was 19-645 days with a mean of 266 days and a median of 133 days. For plasma detection the lead-time range was 27-645 days with a mean of 256 days and a median of 175 days.

In summary, HPVOPC 1) is increasing in incidence, 2) would benefit from improved assessment of disease burden and treatment response, and 3) HPV DNA based testing of salivary rinses and plasma shows promise for assessment of disease burden in HPVOPC affected patients.

### Experiment No. 3

Infection with high-risk human papillomavirus (HPV) is a causative factor for the development of several types of neoplasms, including cervical, anogenital and a subset of head and neck cancers. Cervical carcinoma is one of the most commonly forms of cancer in women worldwide and a leading cause of death among gynecological malignancies. The majority of women diagnosed with cervical cancer exhibit advanced, widely disseminated malignancy and poor prognosis. Greater than 99% of the disease contains HPV sequences which play an important role in the development of cervical cancer precursors. Head and neck squamous cell carcinoma (HNSCC) is the sixth most common cancer worldwide with a mortality rate of 40% to 50% despite aggressive treatment. It's frequently associated with HPV infection, and HPV prevalence is estimated around 30% depending on the anatomic site of the tumor[4]. HPV-driven HNSCC display distinct biological and clinical features, including superior clinical outcomes. HPV positive cancers has less somatic alterations and protein expression change comparing with HPV negative cancers.

After infection, a critical step in progression to cancer is the integration of HPV DNA sequences into the host genome. Integration is associated with alterations in DNA copy number, mRNA transcript abundance and splicing, and both inter- and intrachromosomal rearrangements. Cancers with integrated vs. nonintegrated HPV displayed different patterns of DNA methylation and both human and viral gene expressions. HPV is known to drive cancer by the actions of the E6 and E7 oncoproteins, which would respectively inactivate p53 and members of the pRb family, interfere with the cellular control mechanisms of the cell cycle. However, the functional relevance of other HPV gene products is understudied. In particular, Parfenov et al. found a substantial subset of tumors which demonstrated minimal expression of HPV16 E6 and E7, but dramatic increase in expression of E2, E4, and E5 genes, as well as exclusive association of E2, E4, and E5 expression with lack of HPV16 integration into the host genome. These data imply that there may be an alternate mechanism of HPV viral oncogenesis that does not employ E6 and E7 expression or viral integration, but may be driven by episomal E2, E4, and E5 expression.

In this study, Applicants analyzed HPV gene expression using RNA sequence data in HPV+ HNSCC and CESC and sought to characterize a novel subtype of HPV E2, E4, E5 expressing cancers. To provide functional insights into the HPV16 E2, E4, E5 subtype, Applicants generated upregulation and downregulation experiments in vitro and in vivo, assessed the influence of E2/E4/E5 on biological behavior, and explored possible downstream pathways and targeted molecules. This approach provides a new mechanism in HPV tumorigenesis and characterizes the function of an alternative pathway using non-integrated, E2/E4/E5 expressing HPV as a driver of carcinogenesis.

High risk human papilloma virus related oropharynx carcinoma (HPV OPC) incidence is rapidly increasing, and has surpassed the incidence of cervical carcinoma in the US. Published data show the independent association of post treatment HPV DNA detection in plasma and salivary rinses with HPV OPC recurrence, and concepts in development for NCI supported head and neck clinical trials in HPV OPC currently include research laboratory based assays for detection of HPV DNA in body fluids, including plasma and saliva.

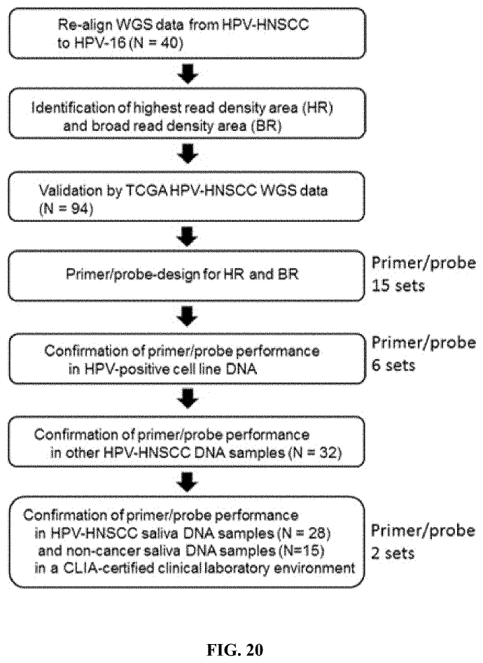

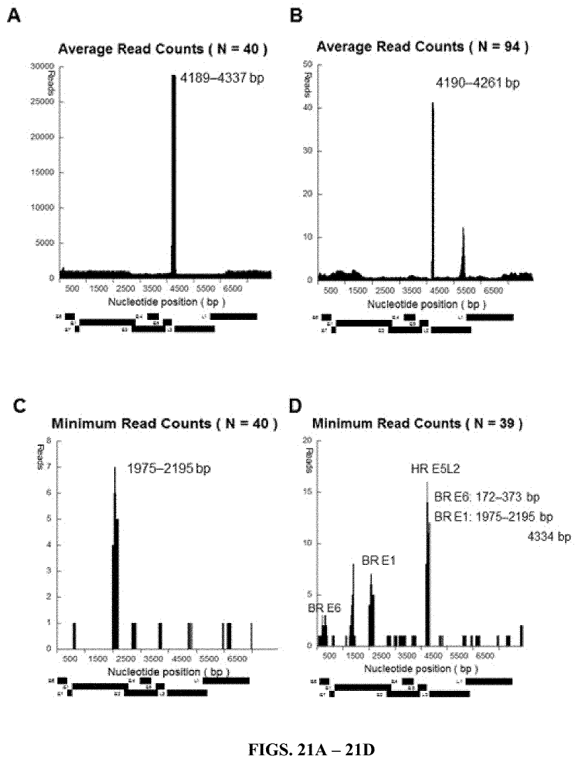

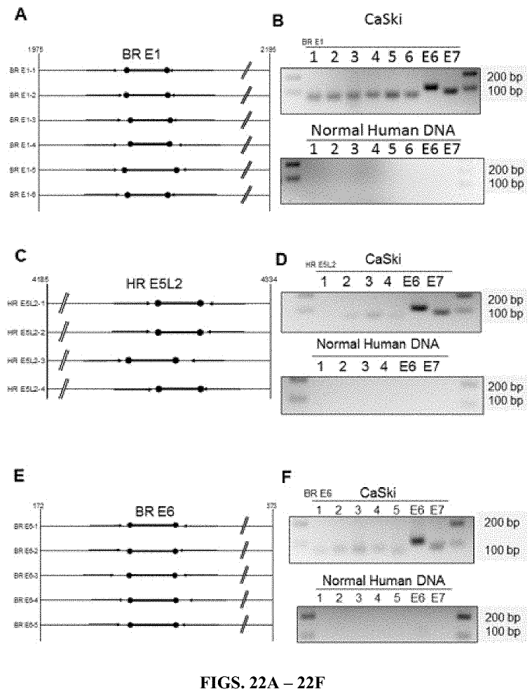

Applicants have developed an HPV DNA based body fluid test based on whole genome sequencing data, followed by analytic validation of this test in plasma and salivary rinses within a CLIA-certified laboratory setting. An additional two months can be added to extend cohort/sample preparation from 4 months to 6 months, to ensure high quality samples can be curated, prepared, and shipped between different institutions. Applicants have designed and analytically validated a quantitative PCR based assay for HPV16 DNA in plasma and salivary rinses using genomic sequencing data from HPV related oropharynx cancer. A summary of the workflow of the same provided in FIG. 20, which shows the stepwise development of the test. To determine the distribution of HPV-16 genome reads in HPV-HNSCC, Applicants performed realignment of the HPV-16 genomic sequence in a discovery set. The discovery set included 40 primary tumor samples from patients with primary OPCs with validated presence of HPV genome. WGS data was used to define the read coverage of each nucleotide position in the HPV-16 genome. Total read count for the HPV-16 genome was 452 590,608 reads. Average read counts of each nucleotide position was evaluated in all 40 samples. Median read for a given nucleotide position was 1019. There were significant spikes (>25 000 reads) in regions extending from 4189 bp to 4337 bp. This result indicated that the region between the E5 and L2 noncoding areas constituted the region of highest read density (HR E5L2) throughout the HPV genome (FIG. 21A).

To validate these findings, Applicants analyzed WGS data from HPV-HNSCC (certain cases were not primary OPCs) in 94 samples from the TCGA cohort (TCGA, Provisional version updated 2016, http://cancergenome.nih.gov/). Clinical records of HPV positivity were obtained from the Broad GDAC Firebrowse website (http://firebrowse.org/). Applicants redefined read coverage for each nucleotide position. Total read count for the HPV-16 genome was 166 026 reads. On analyzing average read counts for each nucleotide position of these 94 patients, it was found that the median read at each nucleotide position was 0.81. Significant spikes (>40 reads) were observed in regions between 4190 bp and 4261 bp at this instance as well. These observations were consistent with the findings from Applicants' “discovery set” cohort (FIG. 21B).

Furthermore, Applicants attempted to define regions where the HPV-16 genome was most likely to appear, across the largest number of tumors, although still at a minimal detectable level in the “discovery set.” Using the 40 tumor samples in the cohort, minimum read counts were plotted for each nucleotide position. Of note, HR E5L2, which had the highest mean read count across the genome, was not universally present in all tumors in Applicants' HPV-16-related HNSCC dataset. Nevertheless, Applicants were able to define a region between 1975 bp to 2195 bp in the E1 region “BR E1” for which every tumor analyzed had a read count of >4 (BR E1) (FIG. 21C). However, Applicants were unable to confirm these observations by TCGA because of low-read coverage data across large parts of the HPV genome in this dataset. As noted, Applicants' discovery cohort coverage (1019 median reads/nucleotide) greatly exceeded that of the TCGA dataset (0.81 median reads/nucleotide.

To identify additional regions with the presence of HPV-16 genome at a minimal detectable level in a large proportion of tumors, Applicants omitted the sample with lowest read coverage (3.92 reads per nucleotide sample) and replotted the minimum read counts for each nucleotide position in the remaining 39 tumor samples. BR E1 remained as the region containing the presence of HPV DNA in all tumors. HR E5L2, along with the entire E6 region (BR E6), was also present in all 39 tumor samples in Applicants' original cohort (FIG. 21D). Applicants' data indicated that these three regions were promising candidate regions for HPV-HNSCC DNA-based detection across the greatest proportion of samples. Further, Applicants investigated the following regions for design of the primer/probe sets: BR E6, 172-373 bp; BR E1, 1975-2195 bp; HR E5L2, 4185-4334 bp.

To optimize HPV DNA primer/probe design and validation for HPV-HNSCC using Primer-BLAST, Applicants constructed candidate primer/probe sets targeting the three regions noted above. PCR was performed using DNA derived from the CaSki cell line as positive control and leukocyte DNA obtained from cancer-free controls as negative control. Applicants created six candidate primer/probe sets (“BR E1-1”-“BR E1-6”) for the BR E1 region (1975-2195 bp) (FIG. 22A, FIG. 22B), four candidate primer/probe sets (“HR E5L2-1”-“HR E5L2-4”) for the HR E5-L2 region (4185-4334 bp) (FIG. 22C, FIG. 22D), and five candidate primer/probe sets (“BR E6-1”-“BR E6-5”) for the BR E6 region (172-373 bp) (FIG. 22E, FIG. 22F). Each of these candidate sets had amplicons (all <80 bp). Positive PCR reactions were observed in the CaSki positive control. No additional primer/probe set (other than those used for minimum read coverage area in the area of regions E7, E2, and L1) was designed because these regions were too limited in size to design any additional appropriate primer probe combinations (FIG. 21C).

In addition, Applicants performed sensitivity analysis to identify the most sensitive primer/probe set from among the candidate sets. First, Applicants confirmed the sensitivity of historical E6 and E7 primer/probe sets; both sets reacted consistently in triplicates to detect <30 copies of HPV-16 genome diluted in samples of normal genomic DNA (10 ng). However, both reacted inconsistently (one or two times of triplicate) to detect 10 copies of HPV-16 genome. Of the 15 designed primer/probe sets, 6 sets consistently amplified under 30 copies of HPV-16 genome. These candidate primer/probe sets included “BR E1-4,” “BR E1-5,” “BR E1-6,” “HR E5L2-4,” “BR E6-1,” and “BR E6-3.” Notably, with the “BR E1-5” primer/probe set, there was consistent detection of 10 copies of HPV-16 genome in triplicate.

To validate the function of primer/probe sets, Applicants calculated the slope of delta Rn against cycle threshold for HPV-16 copy number detection from defined HPV copy number DNA derived from CaSki cell line. Of the six designed primer/probe sets, the values for “BR E1-4,” “BR E1-5,” and “HR E5L2-4” were between −3.58 and −3.10 (Table 2).

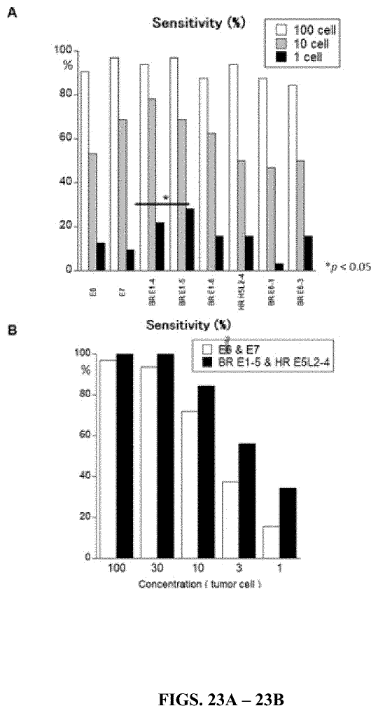

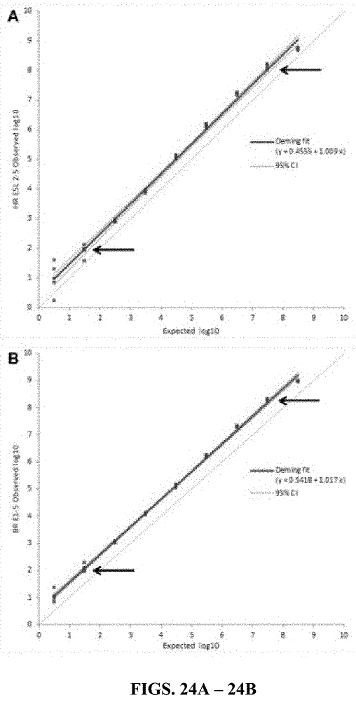

Furthermore, to determine whether these designed primer/probe sets also have a high sensitivity in a separate cohort, Applicants performed PCR in a validation cohort of 32 additional primary tumor samples with validated presence of HPV-16. Applicants prepared serial of dilutions of tumor DNA in normal lymphocyte DNA from non-cancer individuals and determined the threshold for each primer/probe set and defined amplification thresholds expressed in number of tumor cells for each sample (Table 5). In the present study, sensitivity for the detection of ≥100 cells (660 pg DNA) was over 84% for all six designed primer/probe sets and the existing E6/E7 primer/probe sets. In addition, sensitivity for the detection of 10 cells (66 pg DNA) was between 46% and 78% for all primer/probe sets. Furthermore, sensitivity for the detection of DNA from a single cell (6.6 pg DNA) was the highest (28.1%, 9/32) in “BR E1-5” primer/probe sets, followed by “BR E1-4” (21.9%, 7/32), “BR E1-6” (15.6%, 5/32), and “HR E5L2-4” (15.6%, 5/32) primer/probe sets (Table 6). According to the McNemar test, there was a significant difference between the E7 primer set and “BR E1-5” primer/probe sets with respect to single cell detection sensitivity (FIG. 23A). Therefore, the sensitivity of the combination of “BR E1-5” and “HR E5L2-4” was higher than that of E6 and E7 primer/probe sets at each level of tumor dilution (FIG. 23B).

Based on these performance characteristics, Applicants selected E7, “BR E1-5,” and “HR E5L2-4” for further development in a CLIA certified environment.

These primer/probe sets were adapted for development of a CLIA test for HPV16 DNA detection in plasma and salivary rinses (Table 2). Test performance was improved, so that analytical sensitivity in salivary rinse spike in assays ranges from 2.5-5.0 DNA copies/mL with 100% specificity for the three primer/probe sets that are now part of the component assay, resulting in a five-fold improvement in analytic sensitivity that is in the range of sensitivity found in sequence based liquid biopsy methods (FIG. 23, Table 2). Additionally, the assays can be used quantitatively, with measuring ranges between 2.0 log₁₀-7.0 log₁₀ copies/mL.

Of note, Applicants were able to meet goals for reference range, analytical specificity, and precision. Applicants were able to exceed Applicants' goals for limit of detection, reportable/measurable range. Applicants' accuracy showed a Deming regression slope within Applicants' goals. The y-intercept of the Deming regression line was slightly higher than expected, however, this has no adverse effect on ultimate test performance in a clinical setting.

As a final validation, Applicants a small pilot study was performed on a blinded set of saliva rinse samples from 28 patients with HPV-positive HNSCC and 15 salivary rinses from non-cancer controls. Applicants performed sensitivity analysis for E7, “BR E1-5,” and “HR E5L2-4” in saliva rinses. The highest sensitivity for the detection of HPV DNA in saliva rinse samples was observed with E7 (23/28; 82%), followed by “BR E1-5” (22/28; 79%) and “HR E5L2-4” (20/28; 71%). The sensitivity increased to 96% when all three sets were used (Table 3). No signal was noted in non-cancer control patients, yielding a specificity of 100%. This demonstrates that Applicants were able to improve Applicants' sensitivity in a clinical setting from historical reports of approximately 50% to over 90% with no sacrifice in specificity.

### Methods

Identification of HPV Integration and HPV Expression from RNA-Seq Data

Both HPV+ HNSCC and CESC TCGA datasets, as well as an independent primary HPV-positive oropharyngeal squamous cell carcinoma (OPSCC) dataset from Johns Hopkins Hospital (JHH), were used in this study. Methods for sequencing and data processing of RNA using the RNA-seq protocol have been previously described for TCGA and JHH. Integration and expression of HPV genes was identified by taking reads aligned to a combined database of human reference genome and high-risk HPV16, HPV33, HPV35 reference genomes in RNA-seq data using MapSplice (https://github.com/favorov/viruses-in-sequencing). Samples were counted in as HPV-positive by RNA-seq if at least 10 reads mapping to the HPV genome were present. Read counts per HPV gene were determined and collapsed to unique reads per HPV gene (E1, E2, E4, E5, E6, E7, L1 and L2) among variants of HPV. To analyze patterns of differential HPV gene expression, HPV gene read counts were standardized within each tumor (mean=0, SD=1). Then, genes were standardized (mean=0, SD=1). Clustering of these normalized expression values was performed using Rstudio software (https://www.rstudio.com) with the gplots package.

mRNA Expression and DNA Methylation Analysis

Methods for RNA sequencing have been previously described. High density DNA methylation array was used for TCGA Head and Neck samples. The analysis of mRNA expression and DNA methylation datasets was performed using Rtudio software with the gplots package. Applicants carried out unsupervised hierarchical clustering on HPV+ HNSCC TCGA RNA-seq data and JHH RNA-seq data separately using the most variable 1000 expressed genes by comparing standard deviation (SD) among HPV+ tumors and normal samples. Additionally, in HPV+ HNSCC TCGA dataset, Applicants looked for differential expression and methylation between HPV+ HNSCC with high E2, E4, E5 expression or with high E6, E7 expression. For each gene and CpG locus in mRNA expression and DNA methylation datasets, Applicants used significantly differentially expressed genes or methylated CpG loci from Parfenov et al.'s study, respectively.

### Identification of Mutations and CNAs

To characterize mutations and copy number alterations in different HPV genes subtypes, Applicants applied cBioPortal (http://www.cbioportal.org). Top 23 mutations and top 24 CNAs based on frequency in both subtypes of HPV+ HNSCC TCGA dataset were chosen. Removing two duplicate events, Applicants merged mutations and CNAs together to get a 45 differentially variated events list. Frequencies were shown and p values were calculated using Fisher's exact test to compare association between mutations and CNAs and two subtypes. Similarly, top 31 mutations and top 20 CNAs were selected in both subtypes of CESC TCGA dataset. By only keeping unique altered events, Applicants get a 51 differentially alteration events list. Frequencies and p values were also shown by side.

### Survival Analysis

Applicants performed a disease-free survival analysis in a separate HPV-positive OPSCC cohort. Clinical data were reviewed and 23 HPV+ OPSCC patients were included in this prognosis cohort. Recurrence time was defined to be the time in months from tumor biopsy to recurrence, death or loss to follow-up in three years. RT-qPCR using absolute quantitation method was performed to measure the expression levels of each HPV16 genes (E2, E4, E5, E6, E7) in each tumor. 19 tumors had higher expression of E2, E4, E5, while 4 tumors had higher expression of E6, E7. The log rank test was applied to test for the association of HPV subtypes and disease-free survival. 12 samples without recurrence in three years were defined as good prognosis cohort, and 11 samples with recurrence in three years were defined as poor prognosis cohort.

### Quantitative RT-PCR

Total RNA was isolated from tumor tissues and cells using RNeasy Plus Mini Kit (Qiagen). cDNA was obtained using High-Capacity cDNA Reverse Transcription Kit (Applied Biosystems). Quantitative Real-time PCR primers and probes for each HPV genes (E2, E4, E5, E6, E7) were designed using PrimerQuest Tool (https://www.idtdna.com/Primerquest/Home/Index) from Integrated DNA Technologies (IDT) and validated using regular PCR. Optimized primers and probe set were purchased from IDT and sequences of each set are provided in Table 1. RT-qPCR amplification was performed using Platinum Taq DNA Polymerase (Invitrogen). Absolute quantitation method with standard curve was used to measure the exact copy number of each HPV gene in JHH dataset and prognosis cohort. Relative quantification method with comparative CT value was used in cell lines experiments to validate the transfection efficiency. Relative expression levels were normalized to an endogenous control 18S using Hs99999901_s1 18S TaqMan Gene Expression Assays (Applied Biosystems). Error bars indicate SD of three technical replicates and represent at least two independent biological experiments. Statistical analyses were performed by the Student t test.

### Cell Culture

Colon cancer cell line HCT116 p53+/+ was purchased directly from the ATCC and Human Oral Keratinocyte (HOK) was purchased from ScienCell Research Laboratories. Colon cancer cell line HCT116 p53−/− was a gift from Bert Vogelstein's lab (Sidney Kimmel Comprehensive Cancer Center, Johns Hopkins Medical Institutions, Baltimore), cervical cancer cell line Caski was a gift from David Sidransky's lab (Head and Neck Cancer Research, Johns Hopkins Medical Institutions, Baltimore), and HPV-HNSCC cell line Detroit562 and CAL27 were gifts from Silvio Gutkind's lab[25](Moores Cancer Center, University of California, San Diego, La Jolla). HCT116 p53+/+, HCT116 p53−/−, Detroit562 and CAL27 were grown in Dulbecco's Modified Eagle's medium (DMEM, Sigma-Aldrich). Caski was grown in RPMI-1640 Medium (Sigma-Aldrich), and HOK was grown in Oral Keratinocyte Medium (OKM, ScienCell Research Laboratories). All media except OKM were supplemented with 10% FBS (Sigma-Aldrich) and 1% penicillin, streptomycin (Sigma-Aldrich), and cells were cultivated at 37° C. with 5% CO2. Cells were maintained and split every 3 to 4 days according to ATCC recommendations.

Plasmids, Viruses and siRNAs

pCEFL2-E2/4/5 was made by cloning E2/4/5 into pCEFL2 by collaborator in Gutkind's Lab and pCEFL2-E6/7 was produced by recombination pENTR-E6/E7 (Gutkind's Lab) with pCEFL2. Transient transfection was performed using the plasmid of interest, X-tremeGENE 9 DNA Transfection Reagent (Roche) and Opti-MEM Reduced Serum Medium (Gibco). pLenti-E2/E4/E5 was made by recombination pCEFL2-E2/4/5, pENTR and pLenti sequentially, and pLenti-E6/E7 was made by recombination pENTR-E6/E7 and pLenti sequentially. Lentiviruses were produced in 293T cells by cotransfecting pLenti-E2/E4/E5 or pLenti-E6/E7 with the packaging vectors and enveloping vectors using Turbofect (ThermoFisher) according to the manufacturer's instructions. Viral supernatants were collected 72 hours after transfection and concentrated using 20% sucrose in TNE buffer (Tris pH7.5 10 mM, NaCl 150 mM, EDTA 5 mM). Virus-containing pellets were resuspended in TNE buffer and added dropwise on cells in the presence of media supplemented with g/mL polybrene. Selection of lentivirally infected cells was achieved with puromycin used at 1 g/mL. siRNAs targeting each HPV gene (E2, E4, E6, E7) were designed using siDESIGN Center (http://dharmacon.gelifesciences.com/design-center/) from Dharmacon. Downregulation was performed using siRNA of interest, Lipofectamine RNAiMAX Transfection Reagent (ThermoFisher) and Opti-MEM Reduced Serum Medium. RNA collection was employed 48 hours after transient transfection. Overexpression or knockdown efficiency was determined by RT-qPCR.

### Cell Viability Assays

Cells were seeded in five times repeat wells and grown under log phase growth conditions in 96-well plate. After the indicated incubation times, cell viability was measured using the Vita-Blue Cell Viability Reagent (biotool) as described by the manufacturer. Endpoint fluorescence was measured on Synergy HTX multi-mode reader (BioTek). The data displayed are representative of at least three biological experiments performed in five times repeat. Statistical analyses were performed by the Student t test.

### Cell Cycle Assays

Fluorescence-activated cell sorting (FACS) was used to analyze alterations in cell cycle 24 hours after irradiation. Propidium iodide (PI) (Roche) was used as a marker for DNA content. Freshly trypsinized cells were fixed in ice cold 70% ethanol and kept at 4° C. overnight until analysis. Before analysis cells were washed in ice cold PBS and suspended in PBS containing PI (20 g/ml), 0.1% Triton X-100 (Sigma-Aldrich), and RNase A (200 g/ml) (Sigma-Aldrich). Labeled cells were analyzed using a FACSCalibur machine (BD Biosciences). Percentages of cells in each phase of the cell cycle were quantified using the FlowJo software. Statistical analyses were performed by the Student t test.

### Apoptosis Assays

FACS was used to analyze alterations in apoptosis 48 hours after irradiation. Annexin V-FITC Detection Kit (Biotool) was used. Annexin V-FITC labels phosphatidylserine sites on the membrane surface while PI stains necrotic cells. Freshly trypsinized cells were washed in ice cold PBS, suspended in binding buffer and stained with Annexin V-FITC and PI. Labeled cells were analyzed using a FACSCalibur machine (BD Biosciences). Percentages of cells in right upper quadrant and right bottom quadrant were quantified using the FlowJo software. Statistical analyses were performed by the Student t test.

### Transgenic Mice

Tet-E2/E4/E5 mice and Tet-E6/E7 mice were generated by collaborator as previously described. The cK5-rtTA and transgenic FVB/N mice have been previously described. Founders were identified for the presence of the transgene by screening genomic DNA from tail biopsies using a PCR reaction. A similar number of wild-type animals as well as transgenic mice receiving doxycycline treatment were used as controls. Both male and female mice were used in the studies. Doxycycline was administered after birth in the food using grain-based pellets (Bio-Serv) at 6 g/kg.

Single Sample Gene Set Enrichment Analysis (ssGSEA)

Gene set enrichment analysis (GSEA) is the method to determine whether one set of genes are randomly distributed throughout another ranked list of genes or primarily found at the top or bottom. Candidate genes are ranked by their differential expression between two phenotypes. Here Applicants applied an extended version of conventional GSEA in order to produce an enrichment score in a single sample as described previously. Such a score is necessary if one is to make a predictive call on single samples without reference to a larger group of samples. In this approach, the genes are ordered based on either absolute expression or the relative changes with respect to the baseline level. Applicants performed ssGSEA in HPV+ HNSCC TCGA dataset to define differentially expressed gene sets that are unique to E2, E4, E5 expressing tumors as compared to E6, E7 expressing tumors, as well as gene sets expressed in normal samples and subsets of genes that are commonly dysregulated in both sets of HPV tumors. Applicants chose five gene sets significantly expressed in each subset and used Morpheus for visualization and making heatmaps (https://software.broadinstitute.org/morpheus/).

### Results:

### Discovery of HPV Genes Expression Subtypes

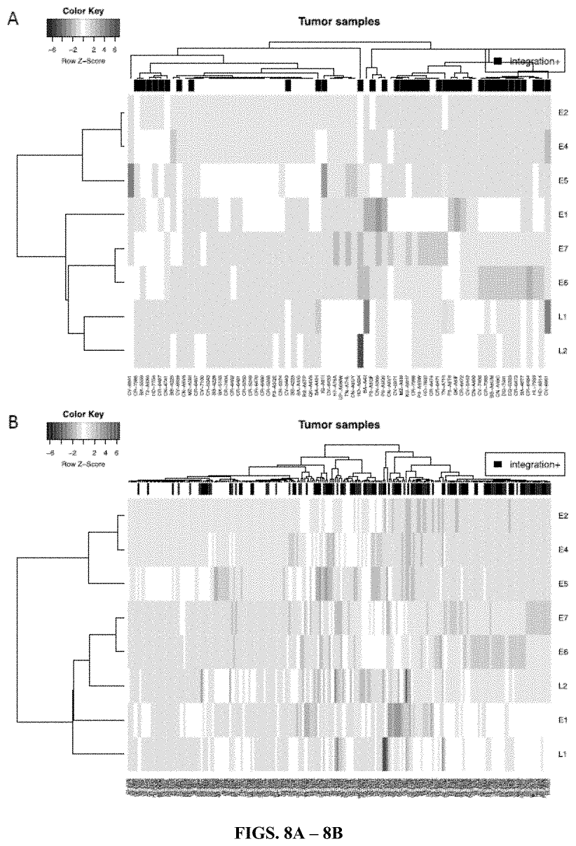

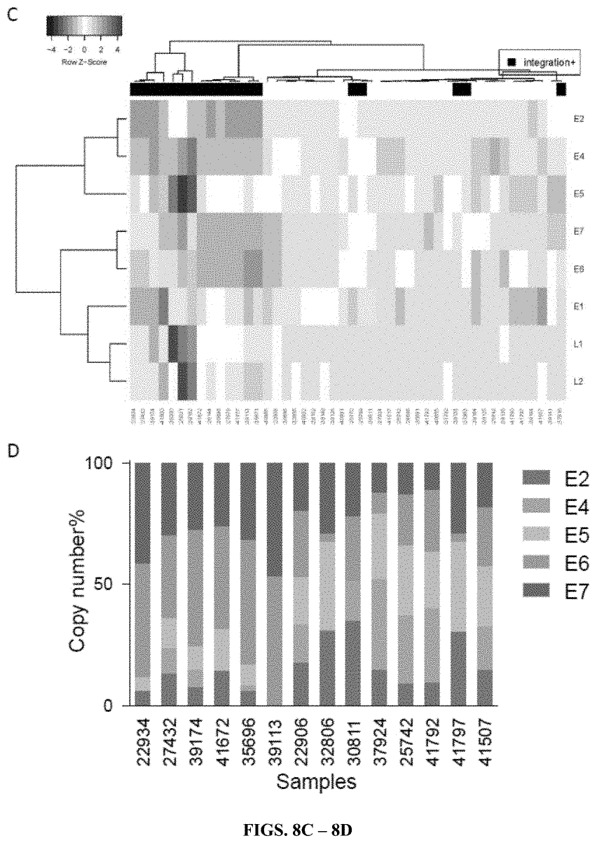

In order to answer the question of whether significant HPV genes expression subtypes exist in HPV+ cancer, Applicants correlated the presence or absence of HPV integration in the host genome with expression of HPV genes. Applicants aligned RNAseq data of HPV+ HNSCC in TCGA to high-risk HPV16, HPV33 and HPV35 genomes independently, and calculated read counts of each gene. Applicants performed unsupervised clustering depending on sum of counts of each HPV gene, and annotated with integration. Applicants found two obvious clusters: tumors with integrated HPV were characterized by high expression of E6, E7 and low expression of E2, E4, E5, while nonintegrated tumors had high expression of E2, E4, E5 and low expression of E6, E7 (FIG. 1A). This association between HPV genes expression and integration status was also found in the TCGA CESC dataset (FIG. 8B) and an independent head and neck (JHH) dataset (FIG. 1C), which were concordant with Parfenov et al. Applicants further validated the RNAseq data by choosing 11 primary tumor samples from JHH dataset and employing qRTPCR using an absolute quantitation method (FIG. 8D) demonstrating complete concordance between RNAseq and qRTPCR data in six samples with high expression in E6, E7, and seven samples with high expression of E2, E4, E5.

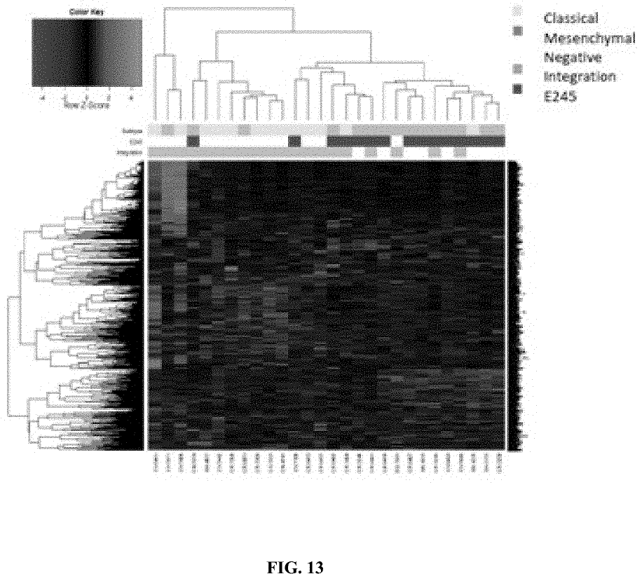

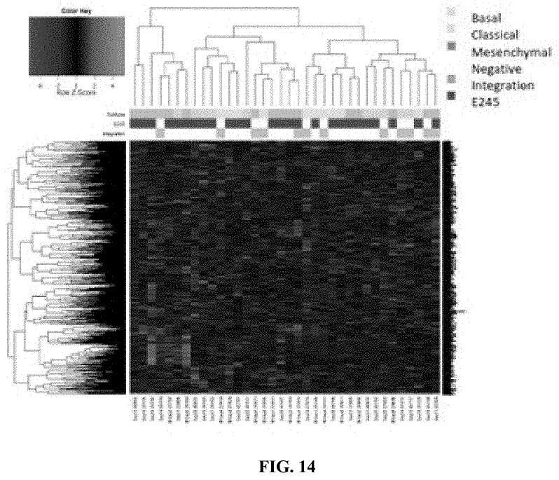

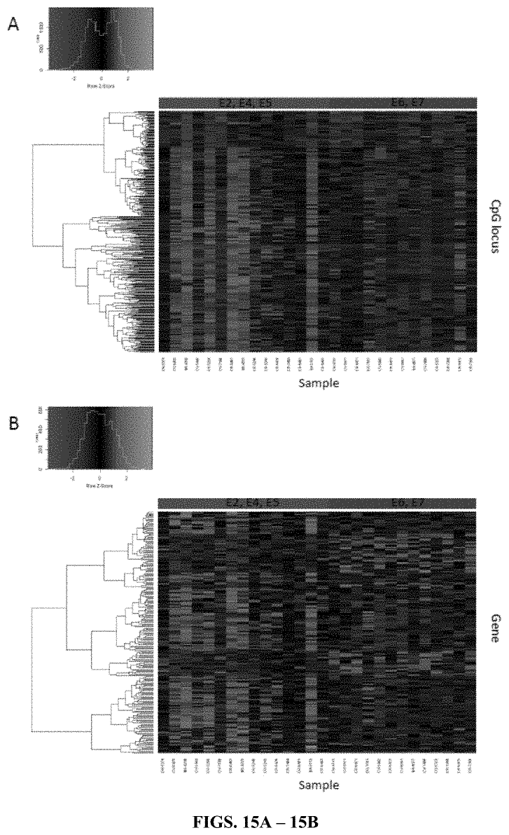

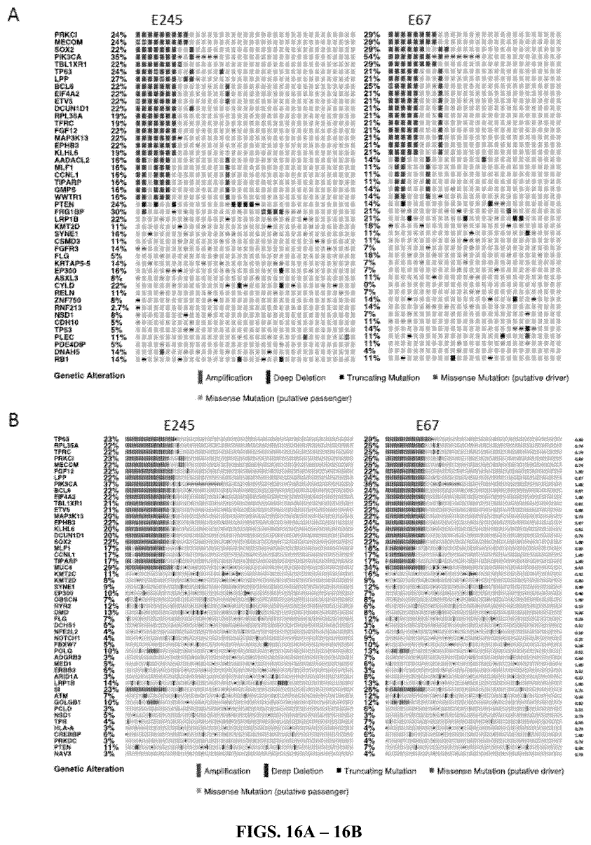

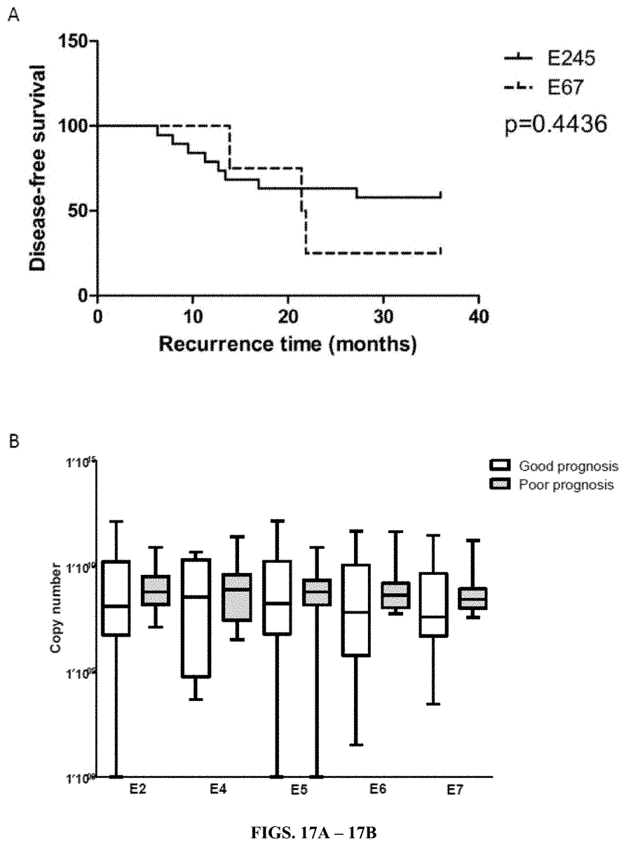

Applicants employed unsupervised clustering of HPV+ HNSCC samples in TCGA dataset (FIG. 13) and JHH dataset (FIG. 14), respectively, using top 1000 genes depending on the standard deviation among HPV+ HNSCC and normal samples. Annotations were made with HPV integration status and genes expression. E2, E4, E5 positive dominated clade tend to have higher expression across these genes than the integration positive dominated clade. Of note, unsupervised clustering of gene expression occurred more strongly according to E2/E4/E5 and E6/E7 expression status, rather than integration, implying that clusters of HPV gene expression drive genomic expression more strongly than integration events. To study the influence of HPV genes expression on the host cell, Applicants compared the patterns of DNA methylation and gene expression of HNSCC in two different HPV genes expression subtypes. Using a linear model of differential methylation and expression for each cytosine-phosphate-guanine (CpG) locus and mRNA, respectively, Applicants found subsets of genes with significantly distinct patterns of methylation (FIG. 15A) and expression (FIG. 15B) among tumors between E2, E4, E5 and E6, E7 subtypes. Applicants chose top mutated genes and copy number alterations (CNA) in each HPV genes subtype and explored the percentages of alteration events in each subtype using cBioPortal (http://www.cbioportal.org) in TCGA HNSCC dataset (FIG. 16A) and CESC dataset (FIG. 16B). p values were calculated using Fisher's test and were listed on the right. Mutations and CNA included SOX2, PIK3CA, FGF12, PTEN, NOTCH1 and ATM, however, there were no differences in mutations and CNA between two subtypes. Furthermore, Applicants investigated the expression of HPV genes subtype with tumor recurrence in patients with HNSCC. There was no statistical association of HPV genes expression with clinical outcome in this relatively small dataset (FIG. 17A), and relatively low expression of E2, E4, E5 as well as E6, E7 was correlated with good prognosis (FIG. 17B).

### E2, E4, E5 Contributes to Cell Proliferation In Vitro and In Vivo

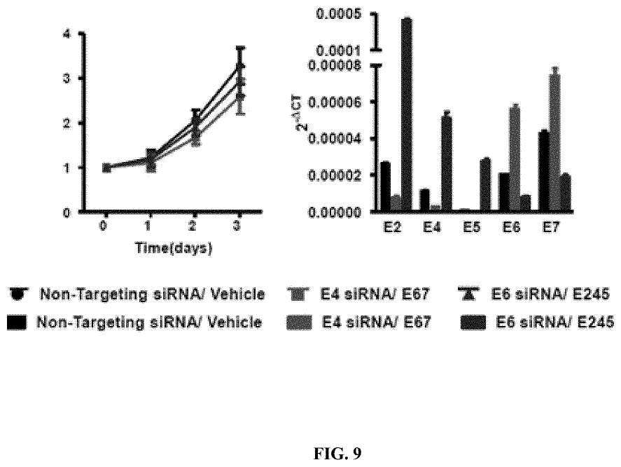

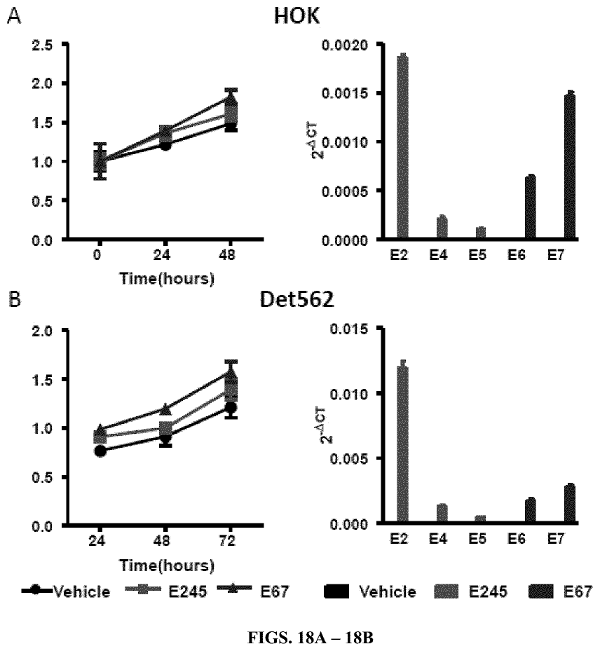

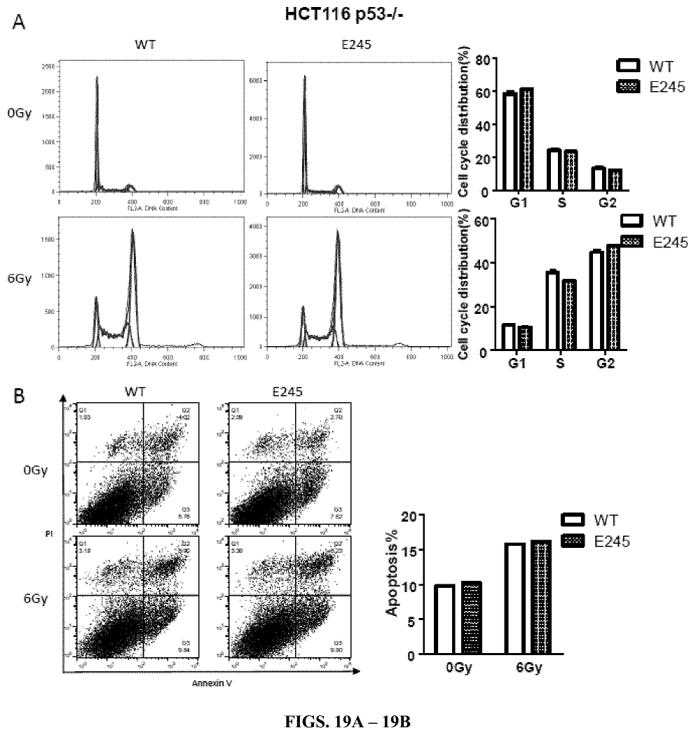

The HPV+ cervical cancer cell line Caski, which expresses all HPV16 genes and contains both episomal and integrated HPV16 genome, was co-transfected with E4 siRNA and pCEFL2 E2/4/5 plasmid or E6 siRNA and pCEFL2 E6/7 plasmid, and cell viability was measured at following 1, 2 and 3 days using vita-blue (FIG. 9, left). Inhibition of E4 or E6 can both suppress the growth of Caski cells regardless of the upregulation of E6/7 or E2/4/5 in the third day comparing with the control group (both p<0.05). RT-qPCR was performed to validate the transfection effect (FIG. 9, right). E2, E4, E5 were essential to the growth of HPV+ cancer cells. Applicants also tested the effect of E2/4/5 or E6/7 overexpression on growth in normal cell line HOK and HPV-HNSCC cell line Detroit, respectively, but Applicants did not find any significant change in growth (FIGS. 18A-B).

Tumor growth induction by E2, E4, E5 in xenograft assays. (FIG. 8B) CAL27/WT, CAL27/E245 and CAL27/E67 cells were implanted in nude mice via subcutaneous injection as described herein.

E2, E4, E5 Reduce Cell Cycle G1 Arrest and Apoptosis after Irradiation in a p53 Dependent Manner

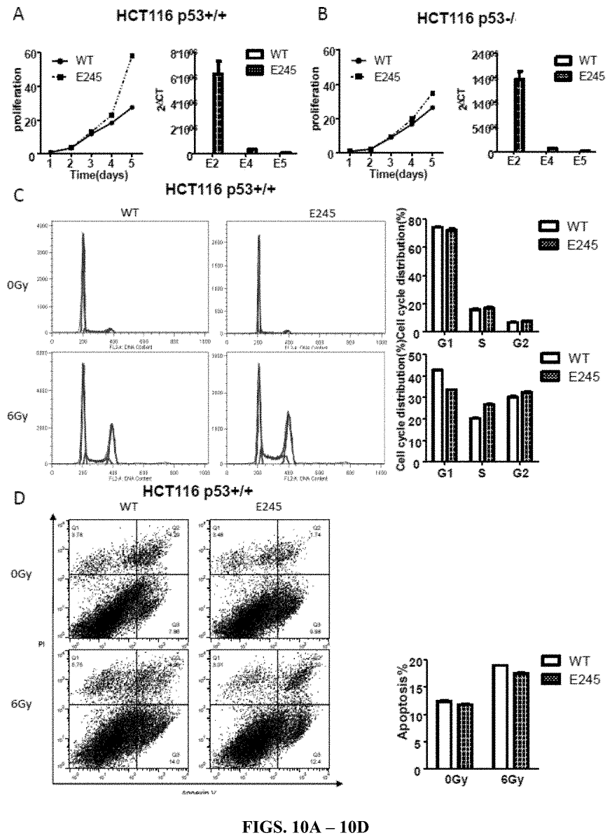

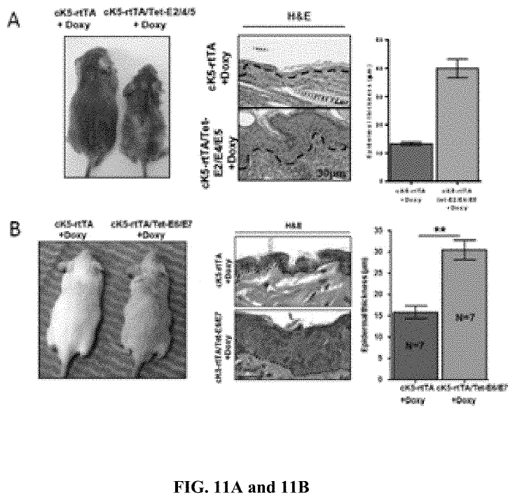

A hallmark of HPV mediated carcinogenesis is inhibition of p53 and Rb through the actions of E6 and E7, respectively. The definition of a clinical HPV cancer phenotype that preferentially works through E2/E4/E5 expression implies that these genes may also affect key cell cycle components and DNA damage response mediators like p53. To explore the hypothesis that E2/E4/E5 expression may also affect p53 function, Applicants chose a well characterized cell line model of p53 function. The HCT116 p53+/+ and p53−/− cell lines were transfected with E2/4/5 overexpressed lentivirus and selected with puromycin. Cell viability was measured at different time-points (1, 2, 3, 4 and 5 days) using vita-blue (FIG. 10A, left; FIG. 10B, left). Both p53+/+ and p53−/− transfected with E2/4/5 showed growth induction compared with control cells. However, growth was more marked in HCT116 p53+/+ compared to p53−/− cells. E2, E4, E5 act on cell viability in a p53 dependent way. RT-qPCR was performed to validate the stable expression of E2, E4, E5 in transfected cells (FIG. 10A, right; FIG. 10B, right).

FACS was used to analyze the cell cycle distribution of irradiated and untreated control cells. PI was incorporated into the cells to stain DNA. Cells were given a dose of 6 Gy and samples were taken at 24 hours following irradiation to monitor cell cycle changes. A rapid increase of irradiated p53+/+ cells in G2 was observed indicating a pronounced G2 arrest. The percentage of cells in the G1-phase of the cell cycle decreased significantly following irradiation in E2/4/5 expressing p53+/+ cells compared with wild type p53+/+ cells, and cells in the S-phase increased significantly, which indicated that a G1-arrest was inhibited by E2/4/5 (FIG. 10C). Irradiated p53−/− cells also initiated an arrest in G2. However, no significant change in E2/4/5 expressing HCT116 p53−/− cells could be seen after irradiation compared with wild type cells (FIG. 19A). This indicates that E2, E4, E5 affect cell cycle and inhibit the G1-arrest after irradiation depending on p53.

The HCT116 p53+/+ and HCT116 p53−/− cells were given a dose of 6 Gy and assayed using FACS at 48 hours after irradiation for detection of apoptosis cells (FIG. 10, FIG. 19B). PI and Annexin V were used stain cells. The percentage of apoptosis cells was estimated by quantification of upper right and bottom right cells using FlowJo. The percentage of apoptosis cells increased dramatically in p53+/+ and p53−/− cells after irradiation. After irradiation, a significant difference between wild type and E2/4/5 expressing p53+/+ cells was observed (vs, p=) (FIG. 10D). No significant change was detected after irradiation in p53−/− cells between two groups (vs, p=) (FIG. 19B). This proves that E2, E4, E5 decrease the percentage of apoptosis cells with the circumstance of p53.

Taken together, these data indicate that HPV16 E2/E4/E5 induce proliferation and cell cycle progression in part through p53 dependent mechanisms, and impair activation of the DNA damage induced G1-S arrest as well as impair induction of apoptosis in a p53 dependent manner.

### Expression of HPV-16 E2/E4/E5 Accelerate Growth In Vivo

To evaluate the effects of HPV-16 E2/E4/E5 expression in the epithelial development and proliferation in vivo, Applicants conditionally expressed the E2/E4/E5 genes by crossing mice carrying cK5 promoter expressing the reverse tetracycline transactivator (rtTA) with mice carrying the E2/E4/E5 genes in the same background under the control of Tet-07 (Tet-E2/E4/E5). Remarkable, Applicants observed a rapid and clear induction of hair loss together with an increase in skin thickness upon Tet activation using doxycycline (FIG. 11A). From previous work by Callejas-Valera et al., an induction of hair loss and an increase of skin thickness were also observed in cK5-rtTA/Tet-E6/E7 mice activated by doxycycline (data not shown). To further explore the ability of E2/E4/E5 in tumor development, Applicants used a classical two-stage carcinogenesis protocol. The carcinogen DMBA causes multiple mutations in the skin and initiates tumorigenesis, and exposure to multiple TPA treatments acts as a promoter. cK5-rtTA/Tet-E2/E4/E5 mice were randomly divided into three groups, DMBA, TPA and DMBA-TPA. HPV16 E2, E4, E5 Expression is Associated with an FGFR Network Activation Signatures

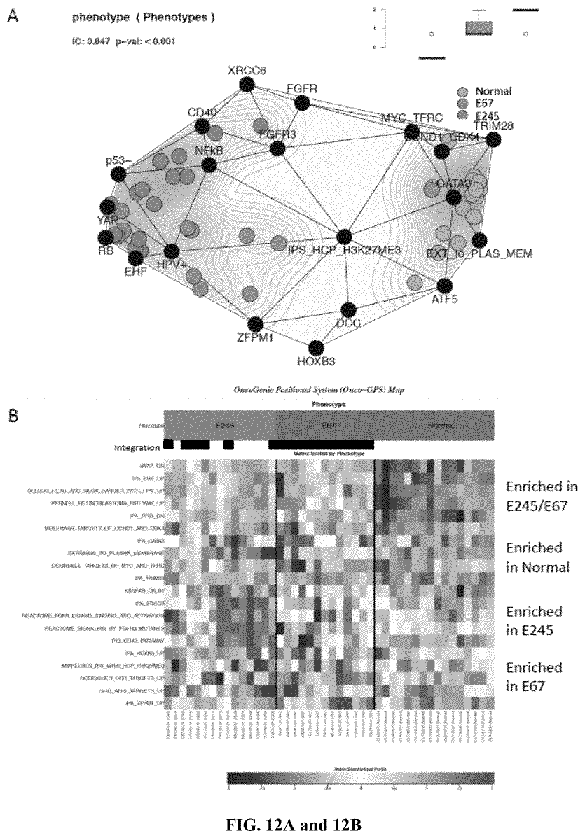

To identify gene sets that correlated with each HPV genes expression subtype, Applicants analyzed RNAseq RSEM in TCGA HPV+ HNSCC using single sample gene set enrichment analysis (ssGSEA) and identified the gene sets most differentially enriched in each subtype, including all HPV+ HNSCC and normal samples. When Applicants compared E2/E4/E5 and E6/E7 subtypes with normal samples, Applicants found 31 overlapped gene sets from top 150 upregulated gene sets, while 13 overlapped gene sets were found from top 150 downregulated gene sets. As Applicants compared E2/E4/E5 subtype with E6/E7 subtype, Applicants found 21 overlapped gene sets from top 500 upregulated gene sets and 27 gene sets from top 500 downregulated gene sets, indicating that these HPV subtypes were closely related, but with distinct network signatures. To provide a visual representation of this relationship, Applicants chose 5 gene sets from each group and drew an OncoGenic Positional System (Onco-GPS) map (FIG. 12A), in which similarity of network expression pattern from one control sample/tumor to another is represented by physical distance. From the onco-GPS map, Applicants noticed that two subtypes share similar relative genes but distinguish from each other. A heatmap of significant gene sets expression in each group was shown (FIG. 12B). Two significant gene sets enriched in E2, E4, E5 subtype are both FGFR gene sets, and the high expression levels of these two gene sets are also associated with non-integration.

### Discussion

Previous studies have established that HPV-driven cancers display distinct biological and clinical features. Many of the hallmark differences identified by existing studies relate to direct action of the viral oncogenes E6 and E7 which have been understood to be the dominant driving force in HPV carcinogenesis. Applicants have found another HPV genes expression subtype in HPV+ tumors—E2/E4/E5 subtype, that is as common or more common in HPV related cervix and head and neck cancer than E6/E7 expressing cancers. Applicants also show that this subtype has biological relevance in vitro and in vivo, and therefore it is a useful and informative classification in HPV+ tumors that complements existing subtypes based on gene expression and integration. Applicants' study points to a critical functional role for E2, E4, E5 in the regulatory landscape of HPV+ cancers. In addition, the E2/E4/E5 subtype is characterized by unique network alterations in oropharyngeal cancers that may be specifically targeted using existing therapeutic agents.

The aetiological role of infection with high-risk HPVs in the vast majority of cervical carcinomas and a substantial proportion of head and neck cancers is well established. HPV16 species group (alpha-9) of the Alpha papillomavirus genus primarily contains HPV16, HPV33, HPV35. These HPVs account for more than 60% of cervical cancers and majority of HPV related head and neck cancers worldwide. Reanalysis of the RNA-seq data of HPV+ HNSCC and CESC in TCGA as well as a separate validation HPV+ OPSCC cohort, clearly show that two clusters—E2, E4, E5 expressing non-integrated cluster and E6, E7 expressing integrated cluster are established. Intuitively, this correlation exists like since, during carcinogenesis parts of HPV DNA (E6, E7) usually integrates into the host genome, leading to loss of parts of HPV DNA including E2, E4, E5. Mixed expression patterns may suggest the presence of both integrated and episomal forms of HPV in the same sample. Unsupervised gene expression clustering of HPV+ HNSCC in TCGA further validated that classification of HPV genes expression is stronger than integration classification. Supervised clustering of DNA methylation levels and mRNA expression levels show that difference between these two HPV gene expression subtypes does exist. However, investigation into mutation, CNA and prognosis of these two subtypes didn't show any significant difference, implying that these global genomic expression differences are more likely driven by HPV gene expression, rather than associated genome mutation, deletion, or amplification.

To further explore the potential pathways and downstream molecules in E2, E4, E5 subtype, Applicants employed ssGSEA and get significant gene sets in E2, E4, E5 subtype, which includes FGFR ligand binding and activation gene set and signaling by FGFR3 mutants gene set. FGFRs, a class of receptor tyrosine kinase (RTK), dimerise and undergo transphosphorylation of the kinase domain upon ligand binding, leading to the recruitment of adaptor proteins and initiating downstream signaling. Because of the ability of FGFR signaling to induce cell proliferation, migration and survival, FGFRs are readily co-opted by cancer cells. In previous study, FGFRs were identified with high mutation incidence in HPV+ HNSCC, and HPV16 E5 induced FGFR2c and triggered epithelial-mesenchymal transition (EMT) in cervical cancer. In Applicants' study, Applicants also show that FGFR expression was upregulated in E2, E4, E5 expression head and neck cell line, and addition of FGFR inhibitor AZD4547 reversed the growth effect induced by E2, E4, E5. The implication of these data are that the HPV E2/E4/E5 subtype of HPV related cancers may be targetable using existing targeted therapeutic strategies, providing a potential option for less toxic or de-escalated therapy for patients with these tumor types.

In conclusion, Applicants found that concurrent expression of HPV16 E2, E4, and E5 genes represents an alternative pathway to HPV related carcinogenesis and defined an alternative mechanism which may provide an opportunity for a novel understanding of HPV carcinogenesis and may allow for rational therapeutic design to interrupt networks involved in cancer development.

TABLE 1
Sequences of primers and probe set of HPV16 genes

| HPV 16 E2 | (SEQ ID NO: | GGAAACACATGCGCCTAGAA |
|---|---|---|
| forward primer | 13) | |
| HPV 16 E2 | (SEQ ID NO: | TCTTTGATACAGCCAGTGTTGG |
| reverse primer | 14) | |
| HPV 16 E2 | (SEQ ID NO: | TGCTATTTATTACAAGGCCAGAG |
| Taqman probe | 15) | AAATGGG |
| HPV 16 E4 | (SEQ ID NO: | CCGAAGAAACACAGACGACTATC |
| forward primer | 16) | |
| HPV 16 E4 | (SEQ ID NO: | TGAGTCTCTGTGCAACAACTTAG |
| reverse primer | 17) | |
| HPV 16 E4 | (SEQ ID NO: | GCGACCAAGATCAGAGCCAGACAC |
| Taqman probe | 18) | |
| HPV 16 E5 | (SEQ ID NO: | ACAACATTACTGGCGTGCT |
| forward primer | 19) | |
| HPV 16 E5 | (SEQ ID NO: | GAGGCTGCTGTTATCCACAATA |
| reverse primer | 20) | |
| HPV 16 E5 | (SEQ ID NO: | GCTTTTGTGTGCTTTTGTGTGT |
| Taqman probe | 21) | CTGCC |
| HPV 16 E6 | (SEQ ID NO: | TCAGGACCCACAGGAGCG |
| forward primer | 22) | |
| HPV 16 E6 | (SEQ ID NO: | CCTCACGTCGCAGTAACTGTTG |
| reverse primer | 23) | |
| HPV 16 E6 | (SEQ ID NO: | CCCAGAAAGTTACCACAGTTATGC |
| Taqman probe | 24) | ACAGAGCT |
| HPV 16 E7 | (SEQ ID NO: | CCGGACAGAGCCCATTACAA |
| forward primer | 25) | |
| HPV 16 E7 | (SEQ ID NO: | CGAATGTCTACGTGTGTGCTTTG |
| reverse primer | 26) | |
| HPV 16 E7 | (SEQ ID NO: | CGCACAACCGAAGCGTAGAGTCAC |
| Taqman probe | 27) | ACT |
| BR E1-1 | (SEQ ID NO: | TGCACAATTGGCAGACACTA |
| forward primer | 28) | |
| BR E1-1 | (SEQ ID NO: | TCCTTTACAATTTTTGCCTGTGA |
| reverse primer | 29) | |
| BR E1-1 probe | (SEQ ID NO: | AGTAATGCAAGTGCCTTTCTAAAA |
| | 30) | AGTA |
| BR E1-2 | (SEQ ID NO: | GCACAATTGGCAGACACTAATA |
| forward primer | 31) | |
| BR E1-2 | (SEQ ID NO: | ATCCTTTACAATTTTTGCCTGTGA |
| reverse primer | 32) | |
| BR E1-2 probe | (SEQ ID NO: | AGTAATGCAAGTGCCTTTCTAAAA |
| | 33) | AGTA |
| BR E1-3 | (SEQ ID NO: | TGCACAATTGGCAGACACTAA |
| forward primer | 34) | |
| BR E1-3 | (SEQ ID NO: | ATCCTTTACAATTTTTGCCTGTGA |
| reverse primer | 35) | |
| BR E1-3 probe | (SEQ ID NO: | TAATGCAAGTGCCTTTCTAAAAAGT |
| | 36) | |
| BR E1-4 | (SEQ ID NO: | ATGCACAATTGGCAGACACTA |
| forward primer | 37) | |
| BR E1-4 | (SEQ ID NO: | ACAATCCTTTACAATTTTTGCCTGT |
| reverse primer | 38) | |
| BR E1-4 probe | (SEQ ID NO: | TAATGCAAGTGCCTTTCTAAAAAGT |
| | 39) | |
| BR E1-5 | (SEQ ID NO: | GCACAATTGGCAGACACTAAT |
| forward primer | 40) | |
| BR E1-5 | (SEQ ID NO: | GCACAATCCTTTACAATTTTTGCC |
| reverse primer | 41) | |
| BR E1-5 probe | (SEQ ID NO: | AGTAATGCAAGTGCCTTTCTAAAA |
| | 42) | AGTAATTC |
| BR E1-6 | (SEQ ID NO: | ATGCACAATTGGCAGACACTAA |
| forward primer | 43) | |
| BR E1-6 | (SEQ ID NO: | AATCCTTTACAATTTTTGCCTGT |
| reverse primer | 44) | GA |
| BR E1-6 probe | (SEQ ID NO: | AGTAATGCAAGTGCCTTTCTAAA |
| | 45) | AAGTA |
| HR E5L2-1 | (SEQ ID NO: | GCAAAACGCACAAAACGTGC |
| forward primer | 46) | |
| HR E5L2-1 | (SEQ ID NO: | GTGGACATGTACCTGCCTGTT |
| reverse primer | 47) | |
| HR E5L2-1 | (SEQ ID NO: | CGGCTACCCAACTTTATAAAACA |
| probe | 48) | |
| HR E5L2-2 | (SEQ ID NO: | TGCAAAACGCACAAAACGTG |
| forward primer | 49) | |
| HR E5L2-2 | (SEQ ID NO: | TGGACATGTACCTGCCTGTTT |
| reverse primer | 50) | |
| HR E5L2-2 | (SEQ ID NO: | CGGCTACCCAACTTTATAAAACA |
| probe | 51) | |
| HR E5L2-3 | (SEQ ID NO: | GCGACACAAACGTTCTGCAA |
| forward primer | 52) | |
| HR E5L2-3 | (SEQ ID NO: | ACCTGCCTGTTTGCATGTTT |
| reverse primer | 53) | |
| HR E5L2-3 | (SEQ ID NO: | CGCACAAAACGTGCATCGGCTA |
| probe | 54) | CCCAAC |
| HR E5L2-4 | (SEQ ID NO: | GCAAAACGCACAAAACGTGC |
| forward primer | 55) | |
| HR E5L2-4 | (SEQ ID NO: | GACATGTACCTGCCTGTTTGC |
| reverse primer | 56) | |
| HR E5L2-4 | (SEQ ID NO: | CGGCTACCCAACTTTATAAAACA |
| probe | 57) | |
| BR E6-1 | (SEQ ID NO: | CAGTTACTGCGACGTGAGGT |
| forward primer | 58) | |
| BR E6-1 | (SEQ ID NO: | ACAGCATATGGATTCCCATCTC |
| reverse primer | 59) | |
| BR E6-1 probe | (SEQ ID NO: | ACTTTGCTTTTCGGGATTTATG |
| | 60) | CATAGTA |
| BR E6-2 | (SEQ ID NO: | ACAGTTACTGCGACGTGAGG |
| forward primer | 61) | |
| BR E6-2 | (SEQ ID NO: | TCACATACAGCATATGGATTCC |
| reverse primer | 62) | CA |
| BR E6-2 probe | (SEQ ID NO: | ACTTTGCTTTTCGGGATTTATGC |
| | 63) | ATAGTA |
| BR E6-3 | (SEQ ID NO: | GCAACAGTTACTGCGACGTG |
| forward primer | 64) | |
| BR E6-3 | (SEQ ID NO: | CACATACAGCATATGGATTCCCA |
| reverse primer | 65) | |
| BR E6-3 probe | (SEQ ID NO: | ACTTTGCTTTTCGGGATTTATGC |
| | 66) | ATAGTA |
| BR E6-4 | (SEQ ID NO: | AAGCAACAGTTACTGCGACG |
| forward primer | 67) | |
| BR E6-4 | (SEQ ID NO: | TCACATACAGCATATGGATTCCC |
| reverse primer | 68) | |
| BR E6-4 probe | (SEQ ID NO: | ACTTTGCTTTTCGGGATTTATGC |
| | 69) | ATAGTA |
| BR E6-5 | (SEQ ID NO: | ACAGTTACTGCGACGTGAGG |
| forward primer | 70) | |
| BR E6-5 | (SEQ ID NO: | CAGCATATGGATTCCCATCTCTA |
| reverse primer | 71) | |
| BR E6-5 probe | (SEQ ID NO: | ACTTTGCTTTTCGGGATTTATGC |
| | 72) | ATAGTA |

TABLE 2
Performance characteristics a CLIA lest for HPV16

| | | Primer-probe region | | |
|---|---|---|---|---|
| Test Characteristic | [symbol] | E7 | HR E5L 2-4 | BR E1-5 |
| Limit of Detection - Plasma | [symbol] HPV16 | 1.5 | 1.5 | 1.5 |
| ( [symbol] /mL) | [symbol] | 13 | 20 | 22 |
| Limit of Detection - Salivary | [symbol] HPV16 | 3 | 2 | 3 |
| rinse ( [symbol] ,/mL) | [symbol] | 23 | 19 | 12 |
| Measuring Range - Plasma | [symbol] HPV16 | 2.0-8.5 log10 | 2.0-8.5 log10 | 2.0-8.5 log10 |
| ( [symbol] /mL) | [symbol] | 2.0-5.0 log10 | 2.0-6.5 log10 | 2.0-6.5 log10 |
| Measurino Range - Salivary | [symbol] HPV16 | 2.0-8.5 log10 | 2.0-8.5log10 | 2.0-8.5 log10 |
| Rinse ( [symbol] /mL) | [symbol] | 2.0-6.5 log10 | 2.0-6.5 log10 | 2.0-6.5 log10 |
| Precision - Plasma (% CV) | [symbol] HPV16 | | | |
| | 2.0 E6 [symbol] /mL | 11% | 28% | 7% |
| | 2.0 E4 [symbol] /mL | 8% | 20% | 13% |
| | 2.0 E2 [symbol] /mL | 25% | 24% | 19% |
| | [symbol] | 5.0 E4 [symbol] /mL, 10% | 2.0 E5 [symbol] /mL, 26% | 2.0 E5 [symbol] /mL, 24% |
| | | 1.0 E2 [symbol] /mL, 11% | 2.0 E3 [symbol] /mL, 55% | 2.0 E3 [symbol] /mL, 23% |
| | | | 2.0 E1 [symbol] /mL, 65% | 2.0 E1 [symbol] /mL, 32% |
| Total Precision - Salivary | [symbol] HPV16 | | | |
| Rinse (% CV) | 8.4 E6 [symbol] /mL | 35% | 39% | 54% |
| | 8.4 E4 [symbol] /mL | 50% | 64% | 50% |
| | 8.4 E2 [symbol] /mL | 53% | 57% | 40% |
| | [symbol] | | | |
| | 2.0 E5 [symbol] /mL | 31% | 21% | 24% |
| | 2.0 E3 [symbol] /mL | 50% | 33% | 43% |
| | 2.0 E1 [symbol] /mL | 62% | 42% | 65% |
| Accuracy - Plasma Blind Panel | Positive % | 100 | 100 | 100 |
| | Agreement | 90 | 100 | 100 |
| | Negative % | 97 | 100 | 100 |
| | Agreement | | | |
| | Total % Agreement | | | |

| | | |
|---|---|---|
| Accuracy - Salivary Rinses | N = 28 salivary rinse | The Triple Test (E7 + HR E5L 2-4 + BR E1-5 results) detected |
| from HNSCC cases | DNAs tested | HPV16 DNA in 27/28 (96%) of salivary rinses |

| | |
|---|---|
| Specificity | No positive results were obtained for any of the 3 primer/probe sets for HIV-1, EBV, |
| | Coxsackie A9, echovirus 11, adenovirus, influenza A virus, herpes simplex virus type |
| | 1, herpes simplex virus type 2, cytomegaiovirus, BK virus: JC virus, human |
| | [symbol] respiratory syncytial virus, human rhinovirus, HCV, HBV, parvovirus |
| | B19 |
| Reference Range | All positive results are reportable |

TABLE 3
Predetermined Metrics and Actual Results

| | | | Performance | |
|---|---|---|---|---|
| Performance | | | Metrics/Milestones: | ACTUAL |
| Characteristic | Experimental Design | Data Analysis | Go/No Go Criteria | RESULT |
| Accuracy | No gold standard quantified | Deming regression | Deming regression line | Y = 1.017x + 0.5 |
| | material exists. In its absence, | analysis of log10- | characteristics: slope | Y = 1.009 + 0.45 |
| | a panel of mock samples | transformed | 1.0 ± 0.1; y-intercept | |
| | (N = 30-50) throughout the | observed vs. | ±0.3 | |
| | measurable range will be | expected results. | | |
| | tested blindly. | | | |
| Precision | Serial dilution of [symbol] DNA | Coefficient of | 40-50% at 100 | 21-57% at 100 |
| | in normal human plasma | variation | copies/mL | [symbol] /ml depending |
| | and salivary rinse samples | | 10-25% at 1000- | on primer probe |
| | 3 levels, 20 replicates on | | 1,000,000 copies/mL | 21-24% at 2.0 E5 |
| | day 1 (intra-run precision) | | | [symbol] /ml depending |
| | 10 addttional replicates on day | | | on primer probe |
| | 2 and day 3 (inter-run | | | |
| | precision) | | | |
| Limit of Detection | Serial dilution of [symbol] DNA | [symbol] regression; | [symbol] , 50-100 copies/mL | 2-20 copies/ml, |
| (“ [symbol] ”, or | in normal human plasma | [symbol] is the | | depending on |
| Analytical | and salivary rinse samples | concentration | | body fluid and |
| Sensitivity) | At least 7 concentrations, 20 | (copies/mL) at | | [symbol] |
| | replicates per concentration in | which 95% of | | |
| | 200 to 500 microliter volume | replicates are | | |
| | range | predicted to be | | |
| | | detected by [symbol] | | |
| | | regression analysis | | |
| Analytical | Salivary rinses from controls | No DNA should be | No DNA should be | NO DNA |
| specificity | and test plasmas from | detectable | detectable | detectable |
| | individuals with other blood- | | | |
| | borne viruses such as but not | | | |
| | limited to HIV, HBV, CMV, | | | |
| | EBV, and adenoviruses | | | |
| Reportable/ | Serial dilution of [symbol] DNA | Deming regression | 100-1,000,000 [symbol] /mL | 2.0-5.0 log10, |
| Measurable range | in normal human plasma | analysis of log10- | | exceeds goal on |
| (only performed | and control salivary rinses | transformed | | both low and high |
| for quantitative | At least 6 concentrations, 5-10 | observed vs. | | limits |
| tests - not | replicates per concentration | expected results. | | |
| qualitative | | Limits of | | |
| detection tests) | | quantification are | | |
| | | defined as highest | | |
| | | and lowest | | |
| | | concentrations with | | |
| | | acceptable | | |
| | | precision (typically | | |
| | | data are within 3- | | |
| | | fold) and linearity | | |
| | | (slope, 0.95-1.0) | | |
| Reference range | Test plasmas arid salivary | No pathogen | No DNA should be | No DNA |
| (aka clinical | rinses from individuals with no | nucleic acid | detectable* | detectable |
| threshold) | history of HNSCC | detected°* | | |
| Harmonization | NA (single laboratory) | NA (single | NA (single laboratory) | NA (single |
| with other | | laboratory) | | laboratory) |
| laboratories | | | | |

TABLE 4
Pilot Data regarding performance of CLIA HPV
test in actual patient and control samples

| Saliva Samples from HPV-HNSCC patients | | | | Saliva Samples from non-tumor patients | | | |
|---|---|---|---|---|---|---|---|
| # | E7 | BR E1-5 | HR E5L2-4 | # | E7 | BR E1-5 | HR E5L2-4 |
| 1 | 328000 | 866000 | 322000 | 1 | N/A | N/A | N/A |
| 2 | 50000 | 118000 | 52500 | 2 | N/A | N/A | N/A |
| 3 | 48500 | 47600 | 25800 | 3 | N/A | N/A | N/A |
| 4 | 46500 | 104000 | 33200 | 4 | N/A | N/A | N/A |
| 5 | 14700 | N/A | N/A | 5 | N/A | N/A | N/A |
| 6 | 7320 | N/A | N/A | 6 | N/A | N/A | N/A |
| 7 | 4950 | 9790 | 9670 | 7 | N/A | N/A | N/A |
| 8 | 1570 | 3590 | 2510 | 8 | N/A | N/A | N/A |
| 9 | 1020 | 2600 | 1590 | 9 | N/A | N/A | N/A |
| 10 | 981 | N/A | N/A | 10 | N/A | N/A | N/A |
| 11 | 521 | N/A | N/A | 11 | N/A | N/A | N/A |
| 12 | 155 | 127 | 86.7 | 12 | N/A | N/A | N/A |
| 13 | 140 | 290 | 53.1 | 13 | N/A | N/A | N/A |
| 14 | 82.7 | 114 | 31.1 | 14 | N/A | N/A | N/A |
| 15 | 62.6 | 160 | 49.7 | 15 | N/A | N/A | N/A |
| 16 | 41.2 | 121 | 41.3 | | | | |
| 17 | 14.1 | 67.4 | 15.8 | | | | |
| 18 | 9.69 | 15.8 | 2.61 | | | | |
| 19 | 8.17 | 61.1 | 26.9 | | | | |
| 20 | 6.5 | 30.9 | 13.4 | | | | |
| 21 | 4.02 | 25.7 | 6.54 | | | | |
| 22 | 2.52 | 2.68 | 1.16 | | | | |
| 23 | 0.986 | 1.82 | N/A | | | | |
| 24 | N/A | 14.4 | N/A | | | | |
| 25 | N/A | 2.76 | N/A | | | | |
| 26 | N/A | N/A | 2.15 | | | | |
| 27 | N/A | 2.17 | 1.19 | | | | |
| 28 | N/A | N/A | N/A | | | | |

TABLE 5
Threshold of tumor cell numbers for HPV-16 detection. The column shown is
the lowest tumor cell counts for the primer/probe for each sample set.

| Sample # | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| E6 | 150.0 | 30.0 | 6.0 | 150.0 | 30.0 | 150.0 | 30.0 | 15.0 | 60.0 | 30.0 | 60.0 | 15.0 | 6.0 | 6.0 | 6.0 | 6.0 |
| E7 | 60.0 | 30.0 | 30.0 | 150.0 | 6.0 | 6.0 | 30.0 | 15.0 | 30.0 | 6.0 | 6.0 | 30.0 | 6.0 | 6.0 | 6.0 | 6.0 |
| BR E1-4 | 150.0 | 150.0 | 1.2 | 6.0 | 30.0 | 30.0 | 30.0 | 50.0 | 1.2 | 1.2 | 1.2 | 3.0 | 6.0 | 6.0 | 6.0 | 6.0 |
| BR E1-5 | 60.0 | 150.0 | 30.0 | 6.0 | 6.0 | 15.0 | 30.0 | 30.0 | 1.2 | 6.0 | 30.0 | 30.0 | 6.0 | 6.0 | 6.0 | 6.0 |
| BR E1-6 | 150.0 | 150.0 | 30.0 | 30.0 | 30.0 | 50.0 | 150.0 | 15.0 | 30.0 | 6.0 | 6.0 | 30.0 | 6.0 | 6.0 | 6.0 | 30.0 |
| HR E5L2-4 | 30.0 | 30.0 | 150.0 | 60.0 | 150.0 | 30.0 | 30.0 | 15.0 | 30.0 | 30.0 | 6.0 | 30.0 | 30.0 | 30.0 | 30.0 | 6.0 |
| BR E6-1 | 150.0 | 30.0 | 150.0 | 60.0 | 30.0 | 60.0 | 30.0 | 150.0 | 30.0 | 30.0 | 30.0 | 15.0 | 60.0 | 60.0 | 30.0 | 6.0 |
| BR E6-3 | 60.0 | 30.0 | 150.0 | 150.0 | 150.0 | 50.0 | 30.0 | 15.0 | 30.0 | 150.0 | 30.0 | 15.0 | 30.0 | 30.0 | 6.0 | 6.0 |

| Sample # | 17 | 18 | 19 | 20 | 21 | 22 | 23 | 24 | 25 | 26 | 27 | 28 | 29 | 30 | 31 | 32 |
|---|---|---|---|---|---|---|---|---|---|---|---|---|---|---|---|---|
| E6 | 30.0 | 6.0 | 1.2 | 0.5 | 1.2 | 0.3 | 1.2 | 1.2 | 0.3 | 0.6 | 15.0 | 3.0 | 3.0 | 15.0 | 3.0 | 15.0 |
| E7 | 1.2 | 6.0 | 1.2 | 1.2 | 1.2 | 1.2 | 1.2 | 1.2 | 0.3 | 0.3 | 1.0 | 1.0 | 3.0 | 15.0 | 0.6 | 15.0 |
| BR E1-4 | 6.0 | 1.2 | 0.3 | 1.2 | 0.6 | 0.3 | 1.2 | 0.6 | 1.2 | 0.6 | 0.6 | 1.0 | 1.0 | 30.0 | 0.6 | 9.0 |
| BR E1-5 | 1.2 | 0.2 | 1.2 | 3.0 | 0.6 | 0.3 | 1.2 | 0.6 | 0.3 | 0.6 | 0.6 | 0.6 | 1.0 | 15.0 | 0.6 | 15.0 |
| BR E1-6 | 6.0 | 1.2 | 1.2 | 6.0 | 1.2 | 0.3 | 1.2 | 1.2 | 1.2 | 0.3 | 0.6 | 0.6 | 3.0 | 150.0 | 0.6 | 9.0 |
| HR E5L2-4 | 6.0 | 6.0 | 1.2 | 1.2 | 6.0 | 0.3 | 1.2 | 1.2 | 0.6 | 0.6 | 0.6 | 1.0 | 3.0 | 30.0 | 0.6 | 30.0 |
| BR E6-1 | 6.0 | 1.2 | 6.0 | 1.2 | 1.2 | 6.0 | 1.2 | 1.2 | 1.2 | 1.2 | 1.0 | 1.0 | 1.0 | 150.0 | 0.6 | 15.0 |
| BR E6-3 | 1.2 | 6.0 | 6.0 | 1.2 | 1.2 | 1.2 | 1.2 | 0.6 | 0.3 | 0.6 | 0.6 | 1.0 | 1.0 | 150.0 | 0.6 | 15.0 |

TABLE 6
Sensitivity from each tumor DNA dilution accoding to the
prime/probe set.

| | Sensitivity | | | | |
|---|---|---|---|---|---|
| Primer/probe set | 100 cells | 30 cells | 10 cells | 3 cells | 1 cell |
| E6 | 0.906 | 0.844 | 0.531 | 0.250 | 0.125 |
| E7 | 0.969* | 0.938* | 0.688 | 0.375 | 0.094 |
| BR E1-4 | 0.938 | 0.875 | 0.781* | 0.531* | 0.219 |
| BR E1-5 | 0.969* | 0.938* | 0.688 | 0.438 | 0.281* |
| BR E1-6 | 0.875 | 0.844 | 0.625 | 0.344 | 0.156 |
| HR E5L2-4 | 0.938 | 0.844 | 0.500 | 0.313 | 0.156 |
| BR E6-1 | 0.875 | 0.750 | 0.469 | 0.344 | 0.031 |
| BR E6-3 | 0.844 | 0.781 | 0.500 | 0.375 | 0.156 |

*showed the highest sensitivity

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Zandberg D P, Bhargava R, Badin S, Cullen K J. The role of human papillomavirus in nongenital cancers. C A Cancer J Clin. 2013; 63(1):57-81.
2. Mork J, Lie A K, Glattre E, Hallmans G, Jellum E, Koskela P, et al. Human papillomavirus infection as a risk factor for squamous-cell carcinoma of the head and neck. The New England journal of medicine. 2001; 344(15):1125-31.
3. Ang K K, Harris J, Wheeler R, Weber R, Rosenthal D I, Nguyen-Tan P F, et al. Human papillomavirus and survival of patients with oropharyngeal cancer. The New England journal of medicine. 2010; 363(1):24-35.
4. Chan K C A, Woo J K S, King A, Zee B C Y, Lam W K J, Chan S L, et al. Analysis of Plasma Epstein-Barr Virus DNA to Screen for Nasopharyngeal Cancer. The New England journal of medicine. 2017; 377(6):513-22.
5. Ahn S M, Chan J Y, Zhang Z, Wang H, Khan Z, Bishop J A, et al. Saliva and plasma quantitative polymerase chain reaction-based detection and surveillance of human papillomavirus-related head and neck cancer. JAMA otolaryngology—head & neck surgery. 2014; 140(9):846-54.
6. Baay M F, Quint W G, Koudstaal J, Hollema H, Duk J M, Burger M P, et al. Comprehensive study of several general and type-specific primer pairs for detection of human papillomavirus DNA by PCR in paraffin-embedded cervical carcinomas. Journal of clinical microbiology. 1996; 34(3):745-7.
7. Wasserman J K, Rourke R, Purgina B, Caulley L, Dimitroulakis J, Corsten M, et al. HPV DNA in saliva from patients with SCC of the head and neck is specific for p16-positive oropharyngeal tumours. Journal of otolaryngology—head & neck surgery=Le Journal d'oto-rhino-laryngologie et de chirurgie cervico-faciale. 2017; 46(1):3.
8. Qureishi A, Ali M, Fraser L, Shah K A, Moller H, Winter S. Saliva testing for human papilloma virus in oropharyngeal squamous cell carcinoma: A diagnostic accuracy study. Clinical otolaryngology: official journal of ENT-U K; official journal of Netherlands Society for Oto-Rhino-Laryngology & Cervico-Facial Surgery. 2017.
9. Marur S, D'Souza G, Westra W H, Forastiere A A. HPV-associated head and neck cancer: a virus-related cancer epidemic. The Lancet Oncology. 2010; 11(8):781-9.
10. Guo T, Sakai A, Afsari B, Considine M, Danilova L, Favorov A V, et al. A Novel Functional Splice Variant of AKT3 Defined by Analysis of Alternative Splice Expression in HPV-Positive Oropharyngeal Cancers. Cancer Res. 2017; 77(19):5248-58.
11. Zehbe I, Wilander E. Two consensus primer systems and nested polymerase chain reaction for human papillomavirus detection in cervical biopsies: A study of sensitivity. Hum Pathol. 1996; 27(8):812-5.
12. Karlsen F, Kalantari M, Jenkins A, Pettersen E, Kristensen G, Holm R, et al. Use of multiple PCR primer sets for optimal detection of human papillomavirus. Journal of clinical microbiology. 1996; 34(9):2095-100.
13. Kalu N N, Mazumdar T, Peng S, Shen L, Sambandam V, Rao X, et al. Genomic characterization of human papillomavirus-positive and -negative human squamous cell cancer cell lines. Oncotarget. 2017; 8(49):86369-83.
14. Nulton T J, Olex A L, Dozmorov M, Morgan I M, Windle B. Analysis of The Cancer Genome Atlas sequencing data reveals novel properties of the human papillomavirus 16 genome in head and neck squamous cell carcinoma. Oncotarget. 2017; 8(11):17684-99.
15. Lydiatt W M, Patel S G, O'Sullivan B, Brandwein M S, Ridge J A, Migliacci J C, et al. Head and Neck cancers-major changes in the American Joint Committee on cancer eighth edition cancer staging manual. C A Cancer J Clin. 2017; 67(2):122-37.
16. Gronhoj Larsen C, Gyldenlove M, Jensen D H, Therkildsen M H, Kiss K, Norrild B, et al. Correlation between human papillomavirus and p16 overexpression in oropharyngeal tumours: a systematic review. Br J Cancer. 2014; 110(6):1587-94.
17. Saito Y, Yoshida M, Omura G, Kobayashi K, Fujimoto C, Ando M, et al. Prognostic value of p16 expression irrespective of human papillomavirus status in patients with oropharyngeal carcinoma. Jpn J Clin Oncol. 2015; 45(9):828-36.
18. Biron V L, Kostiuk M, Isaac A, Puttagunta L, O'Connell D A, Harris J, et al. Detection of human papillomavirus type 16 in oropharyngeal squamous cell carcinoma using droplet digital polymerase chain reaction. Cancer. 2016; 122(10):1544-51.
19. Sidstedt M, Romsos E L, Hedell R, Ansell R, Steffen C R, Vallone P M, et al. Accurate Digital Polymerase Chain Reaction Quantification of Challenging Samples Applying Inhibitor-Tolerant DNA Polymerases. Analytical chemistry. 2017; 89(3):1642-9.
20. Ahn, S. M. et al. Saliva and plasma quantitative polymerase chain reaction-based detection and surveillance of human papillomavirus-related head and neck cancer. JAMA otolaryngology—head & neck surgery 140, 846-854, doi:10.1001/jamaoto.2014.1338 (2014).
21. Hennessey, P. T., Westra, W. H. & Califano, J. A. Human papillomavirus and head and neck squamous cell carcinoma: recent evidence and clinical implications. J Dent Res 88, 300-306 (2009).
22. Parfenov, M. et al. Characterization of HPV and host genome interactions in primary head and neck cancers. Proceedings of the National Academy of Sciences of the United States of America 111, 15544-15549, doi:10.1073/pnas.1416074111 (2014).
23. Ang, K. K. et al. Human papillomavirus and survival of patients with oropharyngeal cancer. The New England journal of medicine 363, 24-35.
24. D'Souza, G. et al. Case-control study of human papillomavirus and oropharyngeal cancer. The New England journal of medicine 356, 1944-1956 (2007).
25. Gillison, M. L. et al. Distinct risk factor profiles for human papillomavirus type 16-positive and human papillomavirus type 16-negative head and neck cancers. Journal of the National Cancer Institute 100, 407-420 (2008).
26. Dahlstrom, K. R., Li, G., Tortolero-Luna, G., Wei, Q. & Sturgis, E. M. Differences in history of sexual behavior between patients with oropharyngeal squamous cell carcinoma and patients with squamous cell carcinoma at other head and neck sites. Head & neck 33, 847-855.
27. Ryerson, A. B. et al. Burden of potentially human papillomavirus-associated cancers of the oropharynx and oral cavity in the US, 1998-2003. Cancer 113, 2901-2909 (2008).
28. Chaturvedi, A. K., Engels, E. A., Anderson, W. F. & Gillison, M. L. Incidence trends for human papillomavirus-related and -unrelated oral squamous cell carcinomas in the United States. Journal of clinical oncology: official journal of the American Society of Clinical Oncology 26, 612-619 (2008).
29. Hammarstedt, L. et al. The incidence of tonsillar cancer in Sweden is increasing. Acta oto-laryngologica 127, 988-992 (2007).
30. Hammarstedt, L. et al. Human papillomavirus as a risk factor for the increase in incidence of tonsillar cancer. International journal of cancer. Journal international du cancer 119, 2620-2623 (2006).
31. Chaturvedi, A. K. et al. Human papillomavirus and rising oropharyngeal cancer incidence in the United States. Journal of clinical oncology: official journal of the American Society of Clinical Oncology 29, 4294-4301.
32. Fakhry, C. & Gillison, M. L. Clinical implications of human papillomavirus in head and neck cancers. Journal of clinical oncology: official journal of the American Society of Clinical Oncology 24, 2606-2611, doi:10.1200/JCO.2006.06.1291 (2006).
33. Lin, J. C. et al. Quantification of plasma Epstein-Barr virus DNA in patients with advanced nasopharyngeal carcinoma. The New England journal of medicine 350, 2461-2470, doi:10.1056/NEJMoa032260 (2004).
34. Strojan, P. et al. Contemporary management of lymph node metastases from an unknown primary to the neck: I. A review of diagnostic approaches. Head & neck.
35. Chuang, A. Y. et al. Presence of HPV DNA in convalescent salivary rinses is an adverse prognostic marker in head and neck squamous cell carcinoma. Oral oncology 44, 915-919(2008).
36. Cao, H. et al. Quantitation of human papillomavirus DNA in plasma of oropharyngeal carcinoma patients. International journal of radiation oncology, biology, physics 82, e351-358.
37. Hennessey, P. T. et al. Serum microRNA biomarkers for detection of non-small cell lung cancer. PloS one 7, e32307.
38. Carvalho, A. L. et al. Evaluation of promoter hypermethylation detection in body fluids as a screening/diagnosis tool for head and neck squamous cell carcinoma. Clinical cancer research: an official journal of the American Association for Cancer Research 14, 97-107(2008).
39. Pattani, K. M. et al. Endothelin receptor type B gene promoter hypermethylation in salivary rinses is independently associated with risk of oral cavity cancer and premalignancy. Cancer Prev Res (Phila Pa.) 3, 1093-1103, doi:1940-6207.CAPR-10-0115 [pii]10.1158/1940-6207.CAPR-10-0115.
40. Carvalho, A. L. et al. Detection of promoter hypermethylation in salivary rinses as a biomarker for head and neck squamous cell carcinoma surveillance. Clinical cancer research: an official journal of the American Association for Cancer Research 17, 4782-4789.
41. Zhao, M. et al. Feasibility of quantitative PCR-based saliva rinse screening of HPV for head and neck cancer. International journal of cancer. Journal international du cancer 117, 605-610 (2005).
42. Schussel, J. et al. EDNRB and DCC salivary rinse hypermethylation has a similar performance as expert clinical examination in discrimination of oral cancer/dysplasia versus benign lesions. Clinical cancer research: an official journal of the American Association for Cancer Research 19, 3268-3275, doi:10.1158/1078-0432.CCR-12-3496 (2013).
43. Caliendo, A. M. et al. Multilaboratory evaluation of real-time PCR tests for hepatitis B virus DNA quantification. Journal of clinical microbiology 49, 2854-2858, doi:10.1128/JCM.00471-11 (2011).
44. Forman, M., Wilson, A. & Valsamakis, A. Cytomegalovirus DNA quantification using an automated platform for nucleic acid extraction and real-time PCR assay setup. Journal of clinical microbiology 49, 2703-2705, doi:10.1128/JCM.00721-11 (2011).
45. Forman, M. S. et al. Diagnostic performance of two highly multiplexed respiratory virus assays in a pediatric cohort. Journal of clinical virology: the official publication of the Pan American Society for Clinical Virology 55, 168-172, doi:10.1016/j.jcv.2012.06.019 (2012).
46. Hirsch, H. H. et al. An international multicenter performance analysis of cytomegalovirus load tests. Clinical infectious diseases: an official publication of the Infectious Diseases Society of America 56, 367-373, doi:10.1093/cid/cis900 (2013).
47. Kanakry, J. A. et al. The clinical significance of EBV DNA in the plasma and peripheral blood mononuclear cells of patients with or without EBV diseases. Blood, doi:10.1182/blood-2015-09-672030 (2016).
48. Kuo, I. C., Espinosa, C., Forman, M. & Valsamakis, A. A polymerase chain reaction-based algorithm to detect and prevent transmission of adenoviral conjunctivitis in hospital employees. Am J Ophthalmol, doi:10.1016/j.ajo.2015.12.007 (2015).
49. Hayden, R. T. et al. Multicenter evaluation of a commercial cytomegalovirus quantitative standard: effects of commutability on interlaboratory concordance. Journal of clinical microbiology 51, 3811-3817, doi:10.1128/JCM.02036-13 (2013).
50. Cancer Genome Atlas, N. Comprehensive genomic characterization of head and neck squamous cell carcinomas. Nature 517, 576-582, doi:10.1038/nature14129 (2015).
51. Greer, A. E., Forman, M. S. & Valsamakis, A. Comparison of BKV quantification using a single automated nucleic acid extraction platform and 3 real-time PCR assays. Diagn Microbiol Infect Dis 82, 297-302, doi:10.1016/j.diagmicrobio.2015.04.006 (2015).
52. Quantitative Molecular Methods for Infectious Diseases; Approved Guideline-Second Edition. CLSI document MM06-A2. (Clinical and Laboratory Standards Institute, Wayne, Pa., USA, 2010).
53. Gillison, M. L. et al. Prevalence of oral HPV infection in the United States, 2009-2010. Jama 307, 693-703, doi:10.1001/jama.2012.101 (2012).
54. Ferlay, J., et al., Estimates of worldwide burden of cancer in 2008: GLOBOCAN 2008. Int J Cancer, 2010. 127(12): p. 2893-917.
55. Siegel, R., et al., Cancer statistics, 2011: the impact of eliminating socioeconomic and racial disparities on premature cancer deaths. C A Cancer J Clin, 2011. 61(4): p. 212-36.
56. Bosch, F. X., et al., The causal relation between human papillomavirus and cervical cancer. J Clin Pathol, 2002. 55(4): p. 244-65.
57. Goon, P. K., et al., HPV & head and neck cancer: a descriptive update. Head Neck Oncol, 2009.1:p.36.
58. Cancer Genome Atlas, N., Comprehensive genomic characterization of head and neck squamous cell carcinomas. Nature, 2015. 517(7536): p. 576-82.
59. Wentzensen, N., S. Vinokurova, and M. von Knebel Doeberitz, Systematic review of genomic integration sites of human papillomavirus genomes in epithelial dysplasia and invasive cancer of the female lower genital tract. Cancer Res, 2004. 64(11): p. 3878-84.
60. Parfenov, M., et al., Characterization of HPV and host genome interactions in primary head and neck cancers. Proc Natl Acad Sci USA, 2014. 111(43): p. 15544-9.
61. Moody, C. A. and L. A. Laimins, Human papillomavirus oncoproteins: pathways to transformation. Nat Rev Cancer, 2010. 10(8): p. 550-60.
62. Callejas-Valera, J. L., et al., mTOR inhibition prevents rapid-onset of carcinogen-induced malignancies in a novel inducible HPV-16 E6/E7 mouse model. Carcinogenesis, 2016. 37(10): p. 1014-25.
63. Barbie, D. A., et al., Systematic RNA interference reveals that oncogenic KRAS-driven cancers require TBK1. Nature, 2009. 462(7269): p. 108-12.
64. Gaykalova, D. A., et al., Novel insight into mutational landscape of head and neck squamous cell carcinoma. PLoS One, 2014. 9(3): p. e93102.
65. Chung, C. H., et al., Molecular classification of head and neck squamous cell carcinomas using patterns of gene expression. Cancer Cell, 2004. 5(5): p. 489-500.
66. Walter, V., et al., Molecular subtypes in head and neck cancer exhibit distinct patterns of chromosomal gain and loss of canonical cancer genes. PLoS One, 2013. 8(2): p. e56823.
67. Ojesina, A. I., et al., Landscape of genomic alterations in cervical carcinomas. Nature, 2014. 506(7488):p.371-5.
68. Crow, J. M., HPV: The global burden. Nature, 2012. 488(7413): p. S2-3.
69. Chen, Z., et al., Evolution and taxonomic classification of human papillomavirus 16 (HPV16)-related variant genomes: HPV31, HPV33, HPV35, HPV52, HPV58 and HPV67. PLoS One, 2011. 6(5): p. e20183.
70. Olthof, N.C., et al., Viral load, gene expression and mapping of viral integration sites in HPV16-associated HNSCC cell lines. Int J Cancer, 2015. 136(5): p. E207-18.
71. Liu, Y., et al., Comprehensive mapping of the human papillomavirus (HPV) DNA integration sites in cervical carcinomas by HPV capture technology. Oncotarget, 2016. 7(5): p. 5852-64.
72. Wesche, J., K. Haglund, and E. M. Haugsten, Fibroblast growth factors and their receptors in cancer. Biochem J, 2011. 437(2): p. 199-213.
73. Seiwert, T. Y., et al., Integrative and comparative genomic analysis of HPV-positive and HPV-negative head and neck squamous cell carcinomas. Clin Cancer Res, 2015. 21(3): p. 632-41.
74. Ranieri, D., et al., HPV16 E5 expression induces switching from FGFR2b to FGFR2c and epithelial-mesenchymal transition. Int J Cancer, 2015. 137(1): p. 61-72.
75. Guo, T., et al., Characterization of functionally active gene fusions in human papillomavirus related oropharyngeal squamous cell carcinoma. Int J Cancer, 2016. 139(2): p. 373-82.
76. Wang, K., et al., MapSplice: accurate mapping of RNA-seq reads for splice junction discovery. Nucleic Acids Res, 2010. 38(18): p. e178.
77. Bibikova, M., et al., High density DNA methylation array with single CpG site resolution. Genomics, 2011. 98(4): p. 288-95.
78. Martin, D., et al., The head and neck cancer cell oncogenome: a platform for the development of precision molecular therapies. Oncotarget, 2014. 5(19): p. 8906-23.
79. Vitale-Cross, L., et al., Conditional expression of K-ras in an epithelial compartment that includes the stem cells is sufficient to promote squamous cell carcinogenesis. Cancer Res, 2004. 64(24): p. 8804-7.
80. Subramanian, A., et al., Gene set enrichment analysis: a knowledge-based approach for interpreting genome-wide expression profiles. Proc Natl Acad Sci USA, 2005. 102(43): p. 15545-50.
81. Tan, Y., et al., Gene signatures related to B-cell proliferation predict influenza vaccine-induced antibody response. Eur J Immunol, 2014. 44(1): p. 285-95.
82. Liberzon, A., et al., The Molecular Signatures Database (MSigDB) hallmark gene set collection. Cell Syst, 2015. 1(6): p. 417-425.

What is claimed is:

1. A method for treating an HPV-related cancer in a patient, the patient having been selected for the treatment by detection of one or more of HPV E2, E4 or E5 in a sample isolated from the patient, the method comprising administering an effective amount of one or more of: FGFR inhibitor AZD4547, radiotherapy, or cisplatin optionally with cetuximab, thereby treating the patient.

2. The method of claim 1, where the patient sample is negative for E6 and E7.

3. The method of claim 1, further comprising detecting one or more of HPV E1, E2, E4, E5, E6 or E7 by contacting the isolated sample with a probe and primer pair of any one of:
a probe and primer pair detecting E1 selected from:
1. a forward primer comprising the sequence: GCACAATTGGCAGACACTAAT, or an equivalent thereof,
2. a reverse primer comprising the sequence: GCACAATCCTTTACAATTTTTGCC, or an equivalent thereof, and
3. a probe comprising the sequence: AGTAATGCAAGTGCCTTTCTAAAAAGTAATTC, or an equivalent thereof,
a probe and primer pair detecting E1 selected from:
a) a forward primer comprising the sequence: GCAAAACGCACAAAACGTGC, or an equivalent thereof,
b) a reverse primer comprising the sequence: GACATGTACCTGCCTGTTTGC, or an equivalent thereof, and
c) a probe comprising the sequence: CGGCTACCCAACTTTATAAAACA, or an equivalent thereof,
a probe and primer pair detecting one or more of HPV E1, E2, E4, E5, E6 or E7 comprising a sequence selected from those listed in Table 1 or an equivalent of each thereof,
a probe and primer pair that detects one of HPV E4 or an equivalent of each thereof, and/or
a probe and primer pair that detects one of HPV E5 or an equivalent of each thereof,
under suitable conditions for detection of any HPV in the sample, and detecting any one of HPV E1, E2, E4, E5, E6 or E7 and, optionally, not detecting any one of HPV E6 or E7 in the sample.

4. The method of claim 1, wherein the one or more of (i) a probe and primer pair that detects one of HPV E2, (ii) a probe and primer pair that detects one of HPV E4, and (iii) a probe and primer pair that detects one of HPV E5 is selected from Table 1.

5. The method of claim 1, wherein the sample is one or more of saliva, blood, plasma, or a tumor sample.

6. The method of claim 1, wherein the patient is a human patient.

7. The method of claim 1, wherein the HPV-related cancer is one or more of HPV-related head and neck squamous cell carcinoma (HNSCC), HPV 16, cancer of the oropharynx, cancer of the pharyngeal, cancer of the cervix, cancer of the vulva, cancer of the penis, cancer of the anus, or cancer of the larynx.

8. A probe and primer pair, comprising:
a) a forward primer comprising the sequence: GCACAATTGGCAGACACTAAT, or an equivalent thereof,
a reverse primer comprising the sequence: GCACAATCCTTTACAATTTTTGCC, or an equivalent thereof, and
a probe comprising the sequence: AGTAATGCAAGTGCCTTTCTAAAAAGTAATTC, or an equivalent thereof, or
b) a forward primer comprising the sequence: GCAAAACGCACAAAACGTGC, or an equivalent thereof,
a reverse primer comprising the sequence: GACATGTACCTGCCTGTTTGC, or an equivalent thereof, and
a probe comprising the sequence: CGGCTACCCAACTTTATAAAACA, or an equivalent thereof.

9. The probe and primer pair of claim 8, further comprising a detectable label on one or more of the forward primer, the reverse primer, or the probe.

10. A method for detecting Human Papilloma Virus (HPV) in a sample comprising contacting the sample with the probe and primer pair of claim 8 and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of the HPV, and detecting any HPV in the sample.

11. The method of claim 10, wherein the one or more of (i) a probe and primer pair that detects one of HPV E2, (ii) a probe and primer pair that detects one of HPV E4, and (iii) a probe and primer pair that detects one of HPV E5 is selected from Table 1.

12. The method of claim 10, wherein the sample is one or more of saliva, blood, plasma, or a tumor sample.

13. A method for detecting Human Papilloma Virus (HPV)-related cancers in a sample comprising contacting the sample with the probe or primer pair of claim 8 and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of the HPV, and detecting any HPV in the sample.

14. The method of claim 13, wherein the one or more of (i) a probe and primer pair that detects one of HPV E2, (ii) a probe and primer pair that detects one of HPV E4, and (iii) a probe and primer pair that detects one of HPV E5 is selected from Table 1.

15. The method of claim 13, wherein the sample is one or more of saliva, blood, plasma, or a tumor sample.

16. A method for monitoring disease progression in a cancer patient in remission from an HPV-related cancer, comprising contacting a sample isolated from the patient with the probe and primer pair of claim 8 and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any HPV in the sample.

17. A method for predicting likelihood of clinical outcome or disease recurrence in a cancer patient, comprising contacting a sample isolated from the patient with the probe and primer pair of claim 8 and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any HPV in the sample.

18. The method of claim 16, wherein the one or more of (i) a probe and primer pair that detects one of HPV E2, (ii) a probe and primer pair that detects one of HPV E4, and (iii) a probe and primer pair that detects one of HPV E5 is selected from Table 1.

19. The method of claim 16, wherein the sample is one or more of saliva, blood, plasma, or a tumor sample.

20. A method of determining whether a cancer patient will benefit from treatment with FGFR inhibitor AZD4547, comprising contacting a sample isolated from the patient with the probe and primer pair of claim 8 and, optionally, one or more of (i) a probe and primer pair that detects one of HPV E2, (ii) a probe and primer pair that detects one of HPV E4, and (iii) a probe and primer pair that detects one of HPV E5, under suitable conditions for detection of any HPV in the sample, and detecting any one of HPV E2, E4, or E5 and, optionally, not detecting any one of HPV E6 or E7 in the sample, wherein such detection indicates a cancer patient will benefit from treatment with FGFR inhibitor AZD4547.


SEQUENCE LISTING (72 sequences)

SEQ ID NO: 1 - DNA, 21 bp, Artificial Sequence
  Description of Artificial Sequence Synthetic primer
gcacaattggcagacactaat

SEQ ID NO: 2 - DNA, 24 bp, Artificial Sequence
  Description of Artificial Sequence Synthetic primer
gcacaatcctttacaatttttgcc

SEQ ID NO: 3 - DNA, 32 bp, Artificial Sequence
  Description of Artificial Sequence Synthetic probe
agtaatgcaagtgcctttctaaaaagtaattc

SEQ ID NO: 4 - DNA, 20 bp, Artificial Sequence
  Description of Artificial Sequence Synthetic primer
gcaaaacgcacaaaacgtgc

SEQ ID NO: 5 - DNA, 21 bp, Artificial Sequence
  Description of Artificial Sequence Synthetic primer
gacatgtacctgcctgtttgc

SEQ ID NO: 6 - DNA, 23 bp, Artificial Sequence
  Description of Artificial Sequence Synthetic probe
cggctacccaactttataaaaca

SEQ ID NO: 7 - DNA, 18 bp, Artificial Sequence
  Description of Artificial Sequence Synthetic primer
tcaggacccacaggagcg

SEQ ID NO: 8 - DNA, 22 bp, Artificial Sequence
  Description of Artificial Sequence Synthetic primer
cctcacgtcgcagtaactgttg

SEQ ID NO: 9 - DNA, 32 bp, Artificial Sequence
  Description of Artificial Sequence Synthetic probe
cccagaaagttaccacagttatgcacagagct

SEQ ID NO: 10 - DNA, 20 bp, Artificial Sequence
  Description of Artificial Sequence Synthetic primer
ccggacagagcccattacaa

SEQ ID NO: 11 - DNA, 23 bp, Artificial Sequence
  Description of Artificial Sequence Synthetic primer
cgaatgtctacgtgtgtgctttg

SEQ ID NO: 12 - DNA, 27 bp, Artificial Sequence
  Description of Artificial Sequence Synthetic probe
cgcacaaccgaagcgtagagtcacact

SEQ ID NO: 13 - DNA, 20 bp, Artificial Sequence
  Description of Artificial Sequence Synthetic primer
ggaaacacatgcgcctagaa

SEQ ID NO: 14 - DNA, 22 bp, Artificial Sequence
  Description of Artificial Sequence Synthetic primer
tctttgatacagccagtgttgg

SEQ ID NO: 15 - DNA, 30 bp, Artificial Sequence
  Description of Artificial Sequence Synthetic probe
tgctatttattacaaggccagagaaatggg

SEQ ID NO: 16 - DNA, 23 bp, Artificial Sequence
  Description of Artificial Sequence Synthetic primer
ccgaagaaacacagacgactatc

SEQ ID NO: 17 - DNA, 23 bp, Artificial Sequence
  Description of Artificial Sequence Synthetic primer
tgagtctctgtgcaacaacttag

SEQ ID NO: 18 - DNA, 24 bp, Artificial Sequence
  Description of Artificial Sequence Synthetic probe
gcgaccaagatcagagccagacac

SEQ ID NO: 19 - DNA, 19 bp, Artificial Sequence
  Description of Artificial Sequence Synthetic primer
acaacattactggcgtgct

SEQ ID NO: 20 - DNA, 22 bp, Artificial Sequence
  Description of Artificial Sequence Synthetic primer
gaggctgctgttatccacaata

SEQ ID NO: 21 - DNA, 27 bp, Artificial Sequence
  Description of Artificial Sequence Synthetic probe
gcttttgtgtgcttttgtgtgtctgcc

SEQ ID NO: 22 - DNA, 18 bp, Artificial Sequence
  Description of Artificial Sequence Synthetic primer
tcaggacccacaggagcg

SEQ ID NO: 23 - DNA, 22 bp, Artificial Sequence
  Description of Artificial Sequence Synthetic primer
cctcacgtcgcagtaactgttg

SEQ ID NO: 24 - DNA, 32 bp, Artificial Sequence; identical to SEQ ID NO: 9
  Description of Artificial Sequence Synthetic probe

SEQ ID NO: 25 - DNA, 20 bp, Artificial Sequence
  Description of Artificial Sequence Synthetic primer
ccggacagagcccattacaa

SEQ ID NO: 26 - DNA, 23 bp, Artificial Sequence
  Description of Artificial Sequence Synthetic primer
cgaatgtctacgtgtgtgctttg

SEQ ID NO: 27 - DNA, 27 bp, Artificial Sequence
  Description of Artificial Sequence Synthetic probe
cgcacaaccgaagcgtagagtcacact

SEQ ID NO: 28 - DNA, 20 bp, Artificial Sequence
  Description of Artificial Sequence Synthetic primer
tgcacaattggcagacacta

SEQ ID NO: 29 - DNA, 23 bp, Artificial Sequence
  Description of Artificial Sequence Synthetic primer
tcctttacaatttttgcctgtga

SEQ ID NO: 30 - DNA, 28 bp, Artificial Sequence
  Description of Artificial Sequence Synthetic probe
agtaatgcaagtgcctttctaaaaagta

SEQ ID NO: 31 - DNA, 22 bp, Artificial Sequence
  Description of Artificial Sequence Synthetic primer
gcacaattggcagacactaata

SEQ ID NO: 32 - DNA, 24 bp, Artificial Sequence
  Description of Artificial Sequence Synthetic primer
atcctttacaatttttgcctgtga

SEQ ID NO: 33 - DNA, 28 bp, Artificial Sequence
  Description of Artificial Sequence Synthetic probe
agtaatgcaagtgcctttctaaaaagta

SEQ ID NO: 34 - DNA, 21 bp, Artificial Sequence
  Description of Artificial Sequence Synthetic primer
tgcacaattggcagacactaa

SEQ ID NO: 35 - DNA, 24 bp, Artificial Sequence
  Description of Artificial Sequence Synthetic primer
atcctttacaatttttgcctgtga

SEQ ID NO: 36 - DNA, 25 bp, Artificial Sequence
  Description of Artificial Sequence Synthetic probe
taatgcaagtgcctttctaaaaagt

SEQ ID NO: 37 - DNA, 21 bp, Artificial Sequence
  Description of Artificial Sequence Synthetic primer
atgcacaattggcagacacta

SEQ ID NO: 38 - DNA, 25 bp, Artificial Sequence
  Description of Artificial Sequence Synthetic primer
acaatcctttacaatttttgcctgt

SEQ ID NO: 39 - DNA, 25 bp, Artificial Sequence
  Description of Artificial Sequence Synthetic probe
taatgcaagtgcctttctaaaaagt

SEQ ID NO: 40 - DNA, 21 bp, Artificial Sequence
  Description of Artificial Sequence Synthetic primer
gcacaattggcagacactaat

SEQ ID NO: 41 - DNA, 24 bp, Artificial Sequence
  Description of Artificial Sequence Synthetic primer
gcacaatcctttacaatttttgcc

SEQ ID NO: 42 - DNA, 32 bp, Artificial Sequence; identical to SEQ ID NO: 3
  Description of Artificial Sequence Synthetic probe

SEQ ID NO: 43 - DNA, 22 bp, Artificial Sequence
  Description of Artificial Sequence Synthetic primer
atgcacaattggcagacactaa

SEQ ID NO: 44 - DNA, 25 bp, Artificial Sequence
  Description of Artificial Sequence Synthetic primer
aatcctttacaatttttgcctgtga

SEQ ID NO: 45 - DNA, 28 bp, Artificial Sequence
  Description of Artificial Sequence Synthetic probe
agtaatgcaagtgcctttctaaaaagta

SEQ ID NO: 46 - DNA, 20 bp, Artificial Sequence
  Description of Artificial Sequence Synthetic primer
gcaaaacgcacaaaacgtgc

SEQ ID NO: 47 - DNA, 21 bp, Artificial Sequence
  Description of Artificial Sequence Synthetic primer
gtggacatgtacctgcctgtt

SEQ ID NO: 48 - DNA, 23 bp, Artificial Sequence
  Description of Artificial Sequence Synthetic probe
cggctacccaactttataaaaca

SEQ ID NO: 49 - DNA, 20 bp, Artificial Sequence
  Description of Artificial Sequence Synthetic primer
tgcaaaacgcacaaaacgtg

SEQ ID NO: 50 - DNA, 21 bp, Artificial Sequence
  Description of Artificial Sequence Synthetic primer
tggacatgtacctgcctgttt

SEQ ID NO: 51 - DNA, 23 bp, Artificial Sequence
  Description of Artificial Sequence Synthetic probe
cggctacccaactttataaaaca

SEQ ID NO: 52 - DNA, 20 bp, Artificial Sequence
  Description of Artificial Sequence Synthetic primer
gcgacacaaacgttctgcaa

SEQ ID NO: 53 - DNA, 20 bp, Artificial Sequence
  Description of Artificial Sequence Synthetic primer
acctgcctgtttgcatgttt

SEQ ID NO: 54 - DNA, 28 bp, Artificial Sequence
  Description of Artificial Sequence Synthetic probe
cgcacaaaacgtgcatcggctacccaac

SEQ ID NO: 55 - DNA, 20 bp, Artificial Sequence
  Description of Artificial Sequence Synthetic primer
gcaaaacgcacaaaacgtgc

SEQ ID NO: 56 - DNA, 21 bp, Artificial Sequence
  Description of Artificial Sequence Synthetic primer
gacatgtacctgcctgtttgc

SEQ ID NO: 57 - DNA, 23 bp, Artificial Sequence
  Description of Artificial Sequence Synthetic probe
cggctacccaactttataaaaca

SEQ ID NO: 58 - DNA, 20 bp, Artificial Sequence
  Description of Artificial Sequence Synthetic primer
cagttactgcgacgtgaggt

SEQ ID NO: 59 - DNA, 22 bp, Artificial Sequence
  Description of Artificial Sequence Synthetic primer
acagcatatggattcccatctc

SEQ ID NO: 60 - DNA, 29 bp, Artificial Sequence
  Description of Artificial Sequence Synthetic probe
actttgcttttcgggatttatgcatagta

SEQ ID NO: 61 - DNA, 20 bp, Artificial Sequence
  Description of Artificial Sequence Synthetic primer
acagttactgcgacgtgagg

SEQ ID NO: 62 - DNA, 24 bp, Artificial Sequence
  Description of Artificial Sequence Synthetic primer
tcacatacagcatatggattccca

SEQ ID NO: 63 - DNA, 29 bp, Artificial Sequence
  Description of Artificial Sequence Synthetic probe
actttgcttttcgggatttatgcatagta

SEQ ID NO: 64 - DNA, 20 bp, Artificial Sequence
  Description of Artificial Sequence Synthetic primer
gcaacagttactgcgacgtg

SEQ ID NO: 65 - DNA, 23 bp, Artificial Sequence
  Description of Artificial Sequence Synthetic primer
cacatacagcatatggattccca

SEQ ID NO: 66 - DNA, 29 bp, Artificial Sequence
  Description of Artificial Sequence Synthetic probe
actttgcttttcgggatttatgcatagta

SEQ ID NO: 67 - DNA, 20 bp, Artificial Sequence
  Description of Artificial Sequence Synthetic primer
aagcaacagttactgcgacg

SEQ ID NO: 68 - DNA, 23 bp, Artificial Sequence
  Description of Artificial Sequence Synthetic primer
tcacatacagcatatggattccc

SEQ ID NO: 69 - DNA, 29 bp, Artificial Sequence
  Description of Artificial Sequence Synthetic probe
actttgcttttcgggatttatgcatagta

SEQ ID NO: 70 - DNA, 20 bp, Artificial Sequence
  Description of Artificial Sequence Synthetic primer
acagttactgcgacgtgagg

SEQ ID NO: 71 - DNA, 23 bp, Artificial Sequence
  Description of Artificial Sequence Synthetic primer
cagcatatggattcccatctcta

SEQ ID NO: 72 - DNA, 29 bp, Artificial Sequence
  Description of Artificial Sequence Synthetic probe
actttgcttttcgggatttatgcatagta